(19) United States
(12) Patent Application Publication
(10) Pub. No.: US 2022/0307055 A1
(43) Pub. Date: Sep. 29, 2022

(54) IMPROVED PRODUCTION OF RECOMBINANT AAV USING EMBRYONATED AVIAN EGGS

(71) Applicant: University of Florida Research Foundation, Incorporated, Gainesville, FL (US)

(72) Inventors: Pedro Cruz, Gainesville, FL (US); Todd Eliot Golde, Gainesville, FL (US); Carolina Ceballos Diaz, South San Francisco, CA (US); Xuefei Liu, Gainesville, FL (US); Daniel Ryu, Gainesville, FL (US)

(73) Assignee: University of Florida Research Foundation, Incorporated, Gainesville, FL (US)

(21) Appl. No.: 17/638,721

(22) PCT Filed: Aug. 28, 2020
(86) PCT No.: PCT/US2020/048632

§ 371 Date: Feb. 25, 2022

Related U.S. Application Data
(60) Provisional application No. 62/893,089, filed on Aug. 28, 2019.

(51) Int. Cl. C12N 15/86; A01K 67/027

(52) U.S. Cl. CPC C12N 15/86; A01K 67/0275; C12N 2750/14122; C12N 2750/14152; C12N 2750/14132; C12N 2710/16644; C12N 2710/10244; C12N 2750/14143; A01K 2217/05; A01K 2227/30

(57) ABSTRACT

Provided herein are improved, cost-effective and environmentally friendly methods of production of recombinant AAV (rAAV) in embryonated avian eggs. Further provided herein is a provides embryonated avian eggs as novel host vehicles for high-yield production of rAAV, including both packaging and propagation. In particular, embryonated chicken eggs provide a novel expression vehicle for AAV of mammalian origin, irrespective of AAV serotype. The disclosed methods may comprise packaging of rAAV in embryonated avian eggs (e.g., chicken eggs) by inoculating an embryonated avian egg with a first nucleic acid vector comprising a transgene and a second nucleic acid vector comprising AAV rep and cap genes, incubating the egg, and isolating rAAV from the egg, wherein the AAV is of non-avian origin. Also provided are methods of purifying and propagating packaged rAAV in embryonated avian eggs or in avian embryonic fibroblasts.

### RELATED APPLICATIONS

This application claims the benefit under 35 U.S.C. § 119(e) of the filing date of U.S. Provisional Application Ser. No. 62/893,089, filed Aug. 28, 2019, the entire contents of which is incorporated herein by reference.

### BACKGROUND

Recombinant adeno-associated viral vectors (rAAV) have become a powerful research and clinical tool due to their ability to provide in vivo long-term gene expression. Recombinant AAV is typically produced in host vehicles such insect cells or mammalian cells. rAAV particle production can involve (1) culturing host cells, (2) introducing AAV genes and any genes desired to be packaged in rAAV particles to the cells, and (3) allowing the cells to produce or package rAAV. The last step is followed by harvesting rAAV particles and subsequent purification steps.

As the uses for rAAV expand, so does the need for large-scale manufacturing methods capable of generating high titers of high quality vector, to not only meet the needs for pre-clinical studies and clinical trials, but the strict quality standards established by the FDA for a gene therapy drug in compliance with current Good Manufacturing Practice (cGMP). Yet the widespread full-scale clinical use of rAAV, in view of commercialization, has long been hampered by manufacturing limitations that have appeared as one of the greatest challenge undermining the production of large quantities of rAAV needed for clinical trials. Current AAV production methods rely on cell batch culturing. Therefore, there is a need for techniques to improve the productivity and yield of large-scale quantities of rAAV particles.

### SUMMARY OF THE INVENTION

The present disclosure provides a novel host vehicle for high-yield production of recombinant AAV, wherein the host vehicle is an embryonated avian egg. The present disclosure provides methods of producing rAAV in embryonated avian eggs that result in improved productivity and yield. These production methods satisfy a need for manufacturing methods with improved yields that are adapted for the scaling necessary for pharmaceutical applications. Advantageously, the production methods disclosed herein are cheaper, more easily scalable and more environmentally friendly than many existing viral vector production methods.

The present disclosure demonstrates that a recombinant AAV of any serotype may be stably packaged and propagated in embryonated avian eggs. The present disclosure also demonstrates that, surprisingly, recombinant AAV may be stably packaged in embryonated avian eggs using the same or similar materials as those used in current mammalian cell-based AAV manufacturing.

Currently, three methods are utilized in the art for rAAV production: transfection, producer cell lines, and viral infection. A majority of transfection protocols rely on double or triple plasmid transfection of adherent human embryonic kidney (HEK293) cells and are considered not scalable due to the linear increase of flat surface for cell culture. Alternatively, stable producer cell line production relies on the introduction of and selection of cells containing either AAV nucleic acids (e.g., AAV rep and cap genes) or the transgene. The main advantage of this method is the increased probability that each cell may result in a rAAV production center, especially when combined with a high transfection or infection efficiency. Among some of the potential issues with cell line derivatives is vector or insert integration loss during prolonged passaging of the cells and the potential for residual adenovirus particles in rAAV products, in cases where an adenovirus is used to provide the AAV rep and cap genes. Further, the AAV Rep protein is known to be genotoxic when stably expressed at high levels. Lastly, methods based on viral infection for rAAV production have been developed utilizing either Baculovirus or Herpes Simplex Virus type 1 (HSV, or HSV1). The concepts of both methods are relatively similar in that rAAV production triggered in the host cells, insect cells or mammalian cells, respectively, upon co-infection with two or more recombinant viruses carrying the various AAV regions, AAV Rep and AAV Cap. However, current yields of AAV grown in insect and mammalian cells are unable to satisfy the demand of industrial scaling.

The present disclosure is based, at least in part, on the inventors' surprising discovery that recombinant AAV of non-avian origin could be stably produced at high yields in embryonated avian eggs. Embryonated avian eggs can be utilized as a novel expression vehicle for production of recombinant non-avian AAV comprising a transgene, e.g., a transgene encoding a therapeutic protein. In particular, embryonated chicken (Gallus gallus) eggs provide a novel expression vehicle for AAV of mammalian origin, irrespective of AAV serotype. The described methods are not only applicable to different AAV serotypes and transgenes to be packaged into rAAV particles, but also compatible with process regulations required for manufacturing of clinical compounds.

Accordingly, the present disclosure provides novel and improved methods of production and preparation, compositions, methods of treatment and expression vehicles relative to the production methods and expression vehicles of the prior art. In 1981, it was shown that primate-derived AAV1 wild-type (wt) stock pre-packaged in mammalian cells could be propagated in embryonated chicken eggs using an avian helper virus known as chicken embryo lethal orphan (CELO) virus. See Petrie & Mayor, J. Virol. Methods, 2: 287-292 (1981), herein incorporated by reference. Petrie & Mayor showed that AAV1 grown in the allantoic fluid of chicken eggs was capable of replication in CV-1 monkey kidney cells and HeLa cells only after co-infection with an SV15 or Ad2 adenovirus helper, respectively. Earlier, Blacklow (J. Natl. Cancer Inst. 40, 19 (1968)) and Ishibashi (Virology 45, 317 (1971)), both of which are herein incorporated by reference, showed that pre-packaged AAV1 wt and AAV2 wt stock could be expressed and propagated in chicken embryo cultures with the use of a CELO helper viral particle. None of these publications suggested that AAV could be packaged in an avian egg, or that an AAV packaged in an avian egg could be subsequently propagated in the same egg.

At the time of Petrie & Mayor's publication, methods and reagents for packaging rAAV by using plasmids (e.g., plasmids containing genes such as AAV rep and AAV cap) had not yet been developed. Transfection of plasmids into embryonated chicken eggs was not developed until 1995 (6). Transfection of plasmids into embryonated chicken eggs have since been performed to produce vaccines (4, 5). Bossis & Chiorini (3) cloned the avian AAV virus (AAAV) and produced recombinant AAAV in HEK293 cells. At around the same time, Grabko & Blyden reported that up to ˜1 mg quantities of recombinant fowl (avian) adenovirus proteins could accumulate in the allantoic fluid of embryonated hen eggs (see International Patent Publication No. WO 2001/19968, incorporated herein by reference). However, none of these publications provided for or suggested that recombinant AAV of non-avian origin (such as AAV of primate origin) could be generated in embryonated avian eggs. There persisted a long absence of recognition in the AAV manufacturing art that AAV may be both packaged and propagated in embryonated avian eggs. Likewise, there was an absence of recognition in the vaccine production art that AAV may be produced in embryonated avian eggs. The present disclosure describes the discovery and validation that AAV may be produced in embryonated avian eggs.

Accordingly, in some aspects, provided herein are methods of producing recombinant AAV (rAAV) comprising: i) inoculating an embryonated avian egg with a first nucleic acid vector comprising a transgene and a second nucleic acid vector comprising AAV rep and cap genes, ii) incubating the egg, and iii) isolating rAAV virions (or particles) from the egg. In these methods, the AAV may be of non-avian origin. The first nucleic acid vector comprises a transgene flanked by AAV inverted terminal repeats, or ITRs. These methods are referred to herein as a “transfection-inoculation protocol.”

In various embodiments, the avian egg is a chicken (Gallus gallus) egg. In some embodiments, the isolated rAAV is substantially free of avian virus material. In various embodiments, the AAV is of mammalian origin (e.g., primate or human origin). See FIG. 6A.

In some embodiments, the disclosed methods further comprise providing one or more helper genes to the embryonated avian egg. Helper genes are important for efficient AAV packaging. The helper genes may be provided in the second nucleic acid vector. The helper genes (or “adenovirus helper genes”) may comprise E1, E2, E4 and VA genes. In such embodiments, the inoculation step may comprise a transfection performed in the presence of a single plasmid, e.g., a plasmid comprising each of the rep, cap, E1, E2, E4 and VA genes. This type of transfection-inoculation is referred to herein as a double transfection protocol.

Alternatively, these helper genes (E1, E2, E4 and VA (RNA) genes) may be provided in a third nucleic acid vector. As such, transfection of the nucleic acid encoding the transgene (e.g., a transgene flanked by AAV ITRs) is performed along with two plasmids (one encoding the rep and cap genes and one encoding the helper genes). In such embodiments, the inoculation step comprise a transfection performed in the presence of two plasmids, e.g., a first plasmid comprising the rep and cap genes and a second plasmid comprising the E1, E2, E4 and VA genes. This type of transfection-inoculation is referred to herein as a triple transfection protocol and a transfection-inoculation protocol. In some embodiments, the transfection is performed using a cationic polymer, cationic lipid, or liposome (e.g., a lipofection), prior to inoculation. In some embodiments, the transfection is performed using a cationic polymer. In particular embodiments, transfection is performed using a polyethylenimine polymer.

In some embodiments, the allantoic cavity or the chorioallantoic membrane of the egg is inoculated one or more nucleic acid vectors (e.g., two, three, or more than three) or packaging viral particles. In certain embodiments, the allantoic cavity is inoculated with first and second nucleic acid vectors. In particular embodiments, the chorioallantoic membrane (CAM) of the egg is inoculated. The CAM is a monolayer of cells surrounding the fluid-filled allantoic cavity of the egg.

In the isolation step, the rAAV may be isolated using a manual pipette or syringe. The rAAV may alternatively be isolated using an automatic, or machine-controlled, pipette or syringe.

In various embodiments, the disclosed methods provide further transfection, propagation and purification steps for the production of purified and/or further concentrated rAAV. For instance, the AAV isolated from the egg as above may be propagated in a second embryonated avian egg for larger scale growth. Propagation in embryonated avian eggs may be used for large-scale viral vector production to be used for manufacturing of medicaments.

The purified and/or concentrated rAAV produced or obtainable by the disclosed methods may be added to a pharmaceutical composition or an rAAV particle. Accordingly, in some aspects, the present disclosure provides compositions comprising purified rAAV or the further purified and/or concentrated rAAV and a pharmaceutically acceptable carrier. Further provided are rAAV particles comprising the purified rAAV or further purified and/or concentrated rAAV.

Further provided herein are methods of treatment comprising administering the compositions and/or rAAV particles described herein to a subject in need thereof. The subject may be a human. The subject may be a patient suffering from a disease, disorder or condition.

In some aspects, the present disclosure provides embryonated avian eggs as a novel host vehicle. In some embodiments, embryonated avian eggs of the disclosure comprise recombinant AAV of mammalian origin, wherein the rAAV comprises a transgene. In particular embodiments, the avian eggs are embryonated chicken eggs. In particular embodiments, the eggs comprise recombinant AAV produced or obtainable by the methods described herein. The transgene may encode a therapeutic peptide.

In other aspects, provided herein are methods of producing recombinant AAV (rAAV) comprising: i) inoculating an embryonated avian egg with a first virus (or viral particle) comprising a transgene and a second virus (or viral particle) comprising AAV rep and cap genes, ii) incubating the egg, and iii) isolating rAAV virions from the egg, wherein the rAAV produced is of non-avian origin. In some embodiments, the first virus (or viral particle) and/or second virus (or viral particle is a Herpes Simplex Virus (HSV) of non-avian origin. The first viral particles comprises the transgene flanked by AAV inverted terminal repeats, or ITRs. In various embodiments, the avian egg is a chicken (Gallus gallus) egg. In some embodiments, the isolated rAAV is substantially free of avian virus material. In various embodiments, the HSV is of mammalian origin (e.g., primate or human origin). The second viral particle may comprise AAV rep2 and/or capX genes (e.g., AAV cap2 or cap9 genes encoding the serotype 2 and serotype 9 capsids, respectively) (see FIGS. 6B and 12). As such, the second viral particle may comprise an rHSV-rep2capX vector (e.g., an rHSV-rep2cap2 or rHSV-rep2cap9 vector), which encodes AAV rep2 and AAV cap2 and cap9, respectively. This methodology is referred to herein as an rHSV inoculation protocol.

In some aspects, a chicken embryo lethal orphan (CELO) virus may be used to deliver the transgene, AAV rep and cap genes, or both. CELO is a type of avian adenovirus. Mammalian HEK293 cells, used in current rAAV manufacturing methods, endogenously express the E1a helper gene for AAV packaging. However, avian eggs do not endogenously express the E1a gene. Thus, CELO vectors may be used in the disclosed methods to supply the E1a gene. Accordingly, in some embodiments, provided herein are methods of producing recombinant AAV (rAAV) comprising: i) inoculating an embryonated avian egg with a first recombinant CELO (rCELO) viral particle comprising a transgene and a second rCELO viral particle comprising AAV rep and cap genes, ii) incubating the egg, and iii) isolating rAAV virions from the egg, wherein the rAAV produced is of non-avian origin. In various embodiments, the avian egg is a chicken egg. In some embodiments, the isolated rAAV is substantially free of avian virus material. This methodology is referred to herein as an rCELO inoculation protocol. In some aspects, provided herein are methods comprising: inoculating an embryonated avian egg with a first recombinant CELO (rCELO) viral particle comprising a transgene and an rHSV comprising AAV rep and cap genes, or vice verse, prior to the step of incubating the egg.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 is a schematic showing a non-limiting example of an AAV packaging method in embryonated avian eggs. A transfection-inoculation protocol is shown, as well as downstream harvesting and purification steps.

FIGS. 2A-2B is a diagram that illustrates the anatomy of an embryonated avian egg. FIG. 2A shows the chalazae, yolk, blastodisc, egg white, airspace, inner shell membrane, outer shell membrane, shell, and cuticle. FIG. 2B shows various routes of injection of recombinant nucleic acid and/or vector into the egg. Injection into the chorioallantoic membrane (CAM) to inoculate the allantoic fluid (allantoic cavity) was found to provide optimal AAV replication.

FIGS. 3A-3C show non-limiting examples of steps of egg inoculation and harvesting. Prior to inoculation, embryonated eggs are candled to determine their stage of development (FIG. 3A). Eggs are inoculated with AAV plasmids by piercing through the shell and CAM using a 20 gauge, 1.5-inch syringe attached to an egg-piercing rubber stopped (FIG. 3B). Eggs are incubated an, once the embryo is determined to be no longer viable by candling, the egg is opened and sterile scissors are used to cut away the shell around the air sac. The allantoic fluid is aspirated using a syringe or a pipette.

FIG. 4 shows results of a proof of concept production (replication) of recombinant AAV-CBA-EGFP genomes in embryonated eggs. Several different AAV serotypes were tested in this experiment: AAV3, AAV4 and AAV9. Purified rAAV-CBA-EGFP virus of low titer was injected (in a volume of 10 μl) into embryonated chicken eggs. Empty (“non-injected”) vector was used as a control. Arrows indicate the presence of rAAV genomes.

FIG. 5 shows results of a proof of concept of the production of rAAV genomes in chicken embryonic fibroblast cells (CEF) at high titer, in the absence of any adenovirus helper. Where no image is shown, very low levels of rAAV production was observed. Arrows indicate the presence of rAAV genomes.

FIGS. 6A-6B are schematics of two exemplary rAAV packaging methods of the disclosure, a) transfection-inoculation protocol and b) rHSV inoculation protocol. In FIG. 6A, a triple transfection-inoculation protocol is shown. Three plasmids—AAV nucleic acid comprising ITRs flanking a transgene, AAV nucleic acid (rep and cap genes), and adenovirus (Ad) helper nucleic acid—are transfected into an embryonated egg. In FIG. 6B, an rHSV inoculation protocol is shown. Two rHSV viral particles—one comprising the transgene and the other comprising AAV nucleic acid (rep and cap genes)—are injected into an egg. In both methods, AAV virus is allowed to replicate in the egg for about 3 days, and rAAV particles are recovered.

FIGS. 7A-7C are images showing high-throughput automatic egg injectors having a production capacity of 62,000 eggs/hour. FIGS. 7A and 7C are photographs of exemplary automatic egg injectors. FIG. 7B depicts a schematic showing piercing of the CAM and inoculation of the allantoic fluid by an automatic injector.

FIG. 8 shows an experimental design for a transfection protocol of an inoculation step of disclosed methods of packaging rAAV particles in embryonated eggs. Transfection involves the combination of a first plasmid encoding a transgene (“Trans”) and a second plasmid encoding a helper nucleic acid (“pHelper”) with PEI, prior to inoculating the PEI/DNA mixture into the allantoic fluid of the egg.

FIG. 9 is a step-by-step diagram of images showing allantoic cavity harvesting after inoculation with rAAV-EGFP operably linked to a chicken β-actin (CBA) promoter. The egg is opened by tapping on the shell just above the air sac, and sterile scissors are used to cut away the shell around the air sac and cut through the egg's chorioallantoic membrane (CAM).

FIG. 10 is a step-by-step diagram of images showing purification of isolated allantoic fluid containing rAAV1 particles by application to discontinuous iodixanol gradient (15% to 54%) and centrifugation. Clarification was performed at 72 hours after transfection. After centrifugation, purified rAAV1 was collected in an Eppendorf tube.

FIGS. 11A-11B show that purified rAAV1 particles produced in embryonated chicken eggs can successfully transduce mammalian cells. FIG. 11A shows EGFP expression after in vitro transduction of mouse primary neuroglia cells (with 5 μl AAV vector), measured by direct observation. FIG. 11B shows EGFP expression following transduction of mouse brain in vivo (with 2 μl AAV vector), as measured by immunohistochemistry. Arrows in FIGS. 11A and 11B indicate the presence of viral transduction in neural cells.

FIG. 12 shows an additional schematic of two examples of rAAV packaging methods of the disclosure, (a) a transfection-inoculation protocol using a polyethylenimine (PEI) cationic polymer and plasmids, and (b) a rHSV transduction-inoculation protocol. The center-panel image shows an inoculation into the allantoic cavity of the embryonated avian egg in accordance with the described methods. In the lower panel, results of an experiment comparing the two protocols is shown. Chorioallantoic membranes (CAM) were analyzed for rAAV packaging potential. The PEI-plasmid transfection efficiency of protocol (a) was analyzed by histochemistry of the red fluorescent protein (RFP) encoded by the pDP2rs plasmid used in this experiment. And the rHSV transduction efficiency of protocol (b) was analyzed by histochemistry of the GFP transgene expression. Arrows indicate successful AAV viral production.

FIG. 13 shows results of allantoic co-inoculation of recombinant rHSV-CBA-hGFP and rHSV-AAV2 (AAV2) into chorioallantoic membrane (CAM) vesicles extracted from embryonated chicken eggs (CAMs numbered 1-3). Results of GFP expression (fluorescence, top) and GFP immunohistochemistry (bottom) are shown. Arrows indicate presence of AAV genomes.

FIG. 14 shows results of allantoic co-inoculation of rHSV-CBA-GFP and rHSV-AAV9 (AAV9) into CAM vesicles extracted from embryonated chicken eggs, CAMs numbered 4-6. Results of GFP expression (fluorescence, top) and GFP immunohistochemistry (bottom) are shown. Arrows indicate presence of AAV genomes.

FIG. 15 shows results of allantoic co-inoculation of rHSV-CBA-GFP and CTR4-EGFP-N1 vectors with a pDP1rs (encodes AAV1 capsids) helper into CAM vesicles extracted from embryonated chicken eggs, CAMs numbered 7-9. Results of GFP expression (fluorescence, top) and GFP immunohistochemistry (bottom) are shown. Arrows indicate presence of AAV genomes.

FIG. 16 shows production of rAAV using CTR4-EGFP-N1, pDP2rs (encodes AAV2 capsids), and rHSV-CBA-hGFP into the CAMs numbered 10-12.

FIG. 17 shows production of rAAV using CTR4-EGFP-N1, pDP6rs (AAV6), and rHSV-CBA-hGFP into the CAMs numbered 13-15.

FIG. 18 shows production of rAAV using CTR4-EGFP-N1, pDP5rs (AAV5), and rHSV-CBA-hGFP into the CAMs numbered 16 and 17.

FIG. 19 shows results of GFP immunohistochemistry negative controls for CAMs 1-3.

FIG. 20 shows results of GFP immunohistochemistry negative controls for CAMs 4-6.

FIGS. 21A-21B show production of AAV using CTR4-EGFP-N1, rHSV-CBA-hGFP, and the helper plasmid pDP1rs, following allantoic inoculation of these plasmids into CAMs of embryonated chicken eggs. FIG. 21A shows results of RFP immunohistochemistry. Arrows indicate presence of AAV genomes. FIG. 21B shows the pDP1rs plasmid map.

FIGS. 22A-22B show production of AAV using CTR4-EGFP-N1, rHSV-CBA-hGFP, and the helper plasmid pDP2rs, following allantoic inoculation of these plasmids into CAMs of embryonated eggs. FIG. 22A shows results of RFP immunohistochemistry. Arrows indicate presence of AAV genomes. FIG. 22B shows the pDP2rs plasmid map.

FIGS. 23A-23B show production of AAV using CTR4-EGFP-N1, rHSV-CBA-hGFP, and the helper plasmid pDP6rs (encodes AAV6 capsid), into CAMs of embryonated eggs. FIG. 23A shows results of RFP immunohistochemistry. FIG. 23B shows the pDP6rs plasmid map.

FIGS. 24A-24B show production of AAV using CTR4-EGFP-N1, rHSV-CBA-hGFP, and the helper plasmid pDP5rs (encodes AAV5 capsid), into CAMs of embryonated eggs. FIG. 24A shows results of RFP immunohistochemistry. FIG. 24B shows the pDP5rs plasmid map.

FIG. 25 shows the plasmid map for CTR4-EGFP-N1.

FIG. 26 shows results of anti-RFP histology (immunochemistry) for CAMs transfected with CTR4-EGFP-N1 and one of the AAV helper plasmids pDP1rs, pDP2rs, pDP5rs, pDP6rs, or without helper plasmid (control).

### DETAILED DESCRIPTION

Provided herein are improved, cost-effective and environmentally friendly methods of production of recombinant AAV (rAAV) in embryonated avian eggs. Further provided herein are embryonated avian eggs as novel host vehicles for high-yield production of rAAV, including both packaging and propagation. In particular, embryonated chicken eggs provide a novel expression vehicle for AAV of mammalian origin, irrespective of AAV serotype. The present disclosure provides for stable packaging of rAAV in embryonated avian eggs using the same materials as those used in current mammalian cell-based AAV manufacturing, such as HEK293 cell-based manufacturing. That is, AAV may be stably packaged through a transfection of as few as two plasmids that together comprise all genes necessary for efficient packaging, rather than through multiple plasmids or viral particles. Unlike in prior publications, these recombinant AAV viruses are of non-avian origin (e.g., they do not comprise avian AAV and may not contain any avian genetic components). The present disclosure describes the discovery and validation that AAV particles, including AAV particles encoding a therapeutic transgene, may be both packaged and propagated in a single embryonated avian egg.

As the effectiveness of rAAV-based therapies becomes more evident by clinical outcomes in a number of indications, so does the demand for improved scalable, productive and high-yield producing methods of rAAV particle production. Furthermore, the need for these rAAV production methods to be GLP/GMP-compatible is pertinent for end use as clinical compounds.

As such, provided herein are methods of packaging AAV vector in embryonated avian eggs, such as embryonated chicken eggs. The packaging comprises an inoculation step, which in some embodiments may comprise a transfection method. The described methods may make use of one or more nucleic acid vector plasmids, such as two or more plasmids. Alternatively, they may make use of one or more recombinant herpes simplex virus (rHSV) particles, such as two rHSV particles. In some embodiments, the disclosed methods make use of one or more adenovirus particles. In some embodiments, the disclosed methods make use of one or more CELO virus particles (which resemble avian adenovirus particles).

In some embodiments, viruses other than AAV may be packaged and/or propagated in accordance with the methods of this disclosure.

The disclosed methods of producing rAAV may comprise: i) inoculating an embryonated avian egg with a first nucleic acid vector comprising a transgene and a second nucleic acid vector comprising AAV rep and cap, ii) incubating the egg, and iii) isolating rAAV virions (or particles) from the egg. In some embodiments, the produced AAV virus is of non-avian origin. In particular embodiments, the AAV virus is of primate origin, such as human origin or non-human primate origin.

As used herein, the term “nucleic acid vector” embraces any nucleic acid molecule, for instance a plasmid, e.g., a DNA plasmid. In various embodiments, the first, second and/or third nucleic acid vectors of the presently disclosed methods are DNA plasmids.

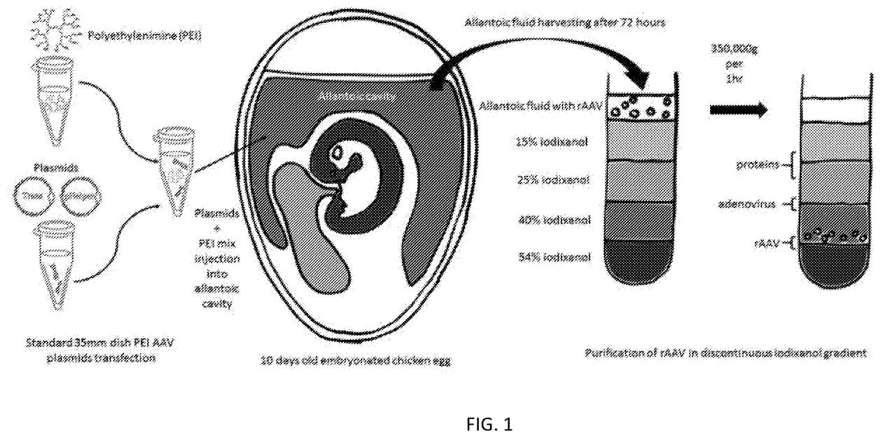

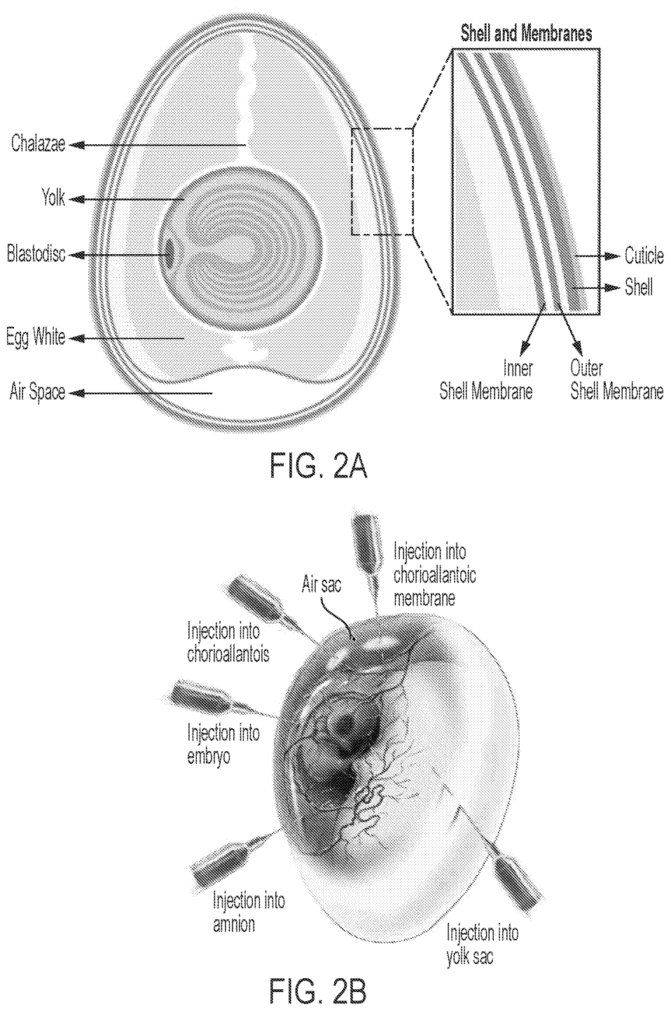

As used herein, the term “embryonated avian egg” refers to a fertilized egg of an avian species in which an embryo has formed, wherein the egg contains an allantoic cavity, an amnion, and a yolk sac (see FIGS. 1, 2A, and 2B). The disclosure embraces embryonated avian eggs of various species, such as chicken eggs and goose eggs.

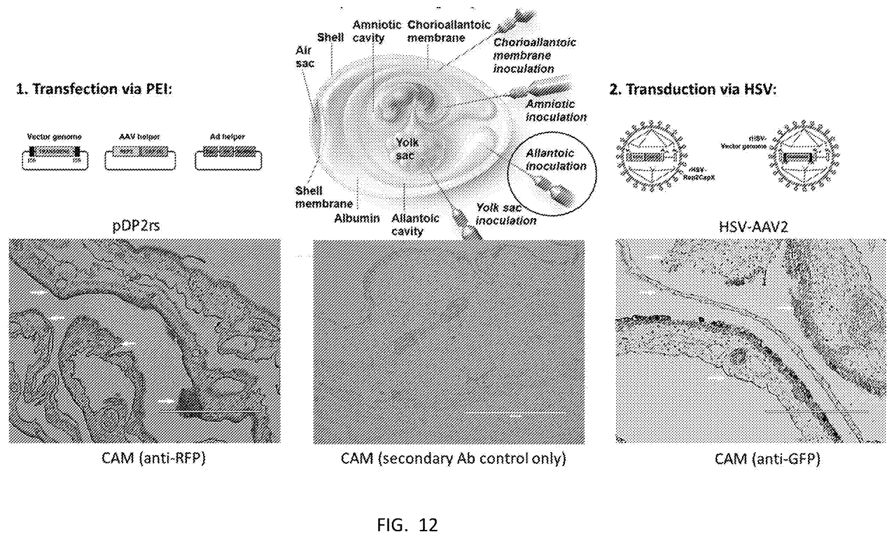

AAV genes and any genes desired to be packaged into rAAV particles may be introduced to cells by either transfection methods (e.g., using plasmid vectors and a transfection agent) or infection methods (e.g., using a viral vector). Cells are said to be “transfected” or “infected” at the time when transfection or infection reagents (e.g., vectors) are first introduced to the cells. In certain embodiments of the disclosed methods, the first and second nucleic acid vectors are transfected with a cationic polymer prior to inoculation. The cationic polymer may comprise polyethylenimine (PEI) (see FIGS. 8 and 12).

In some embodiments, the chorioallantoic membrane (CAM) of the egg is inoculated. In some embodiments, the allantoic cavity or fluid of the egg is inoculated. This may be referred to herein as an “allantoic inoculation.” In other embodiments, the amnion or yolk sac is inoculated. In some embodiments, the allantoic cavity or the chorioallantoic membrane of the egg is inoculated with the first and second nucleic acid vectors (see FIGS. 2A, 2B, 7B, and 12).

In certain embodiments, the egg to be inoculated is 10 days old, as determined by candling. In other embodiments, the egg to be inoculated is 8 days old, 9 days, old, 11 days old, 12 days old, 13 days old, or 15 days old.

The avian egg may be incubated for a period of time following transfection but before harvesting. In some embodiments, the egg is incubated for a period of at least 15 hours, at least 20 hours, at least 24 hours, at least 36 hours, at least 54 hours, at least 60 hours, at least 72 hours, at least 80 hours, or at least 90 hours. In particular, the egg may be incubated for about 72 hours. Alternatively, the egg may be incubated for about 54 hours, about 60 hours, about 66 hours, about 78 hours or about 84 hours.

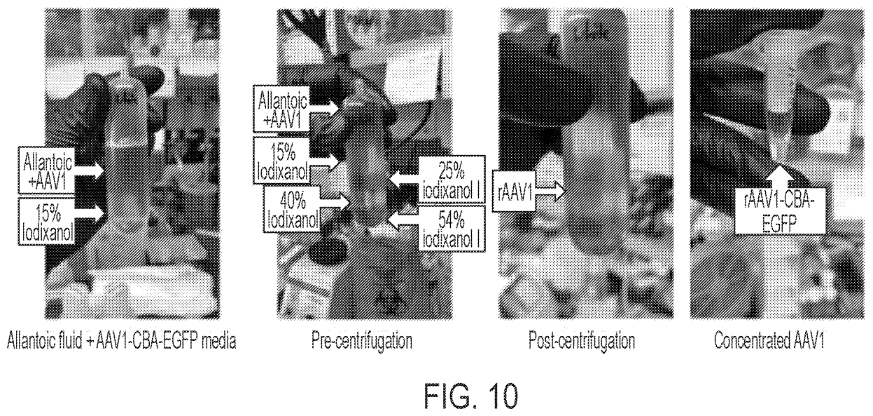

Further disclosed are methods of purifying rAAV virions isolated from embryonated avian eggs. In particular embodiments, the methods comprise subjecting the isolated rAAV to an iodixanol gradient and/or affinity chromatography. A discontinuous iodixanol gradient (e.g., a gradient of between 15% and 54%) may be used for these methods (see FIGS. 1 and 10). In some embodiments, an iodixanol gradient is used wherein the lowest concentration of iodixanol in the gradient is 15%. Additional affinity purification methods that may be used with the rAAV virions on this disclosure are disclosed in U.S. Publication No. 2017/0130208, herein incorporated by reference. The resulting separated mixture may be centrifuged (e.g., at 350,000 g) for about 1 hour to isolate rAAV (see FIG. 1).

In certain embodiments, the disclosed production methods further comprise propagating and generating purified rAAV particles. In some embodiments, avian eggs (e.g., chicken eggs) are used as a host vehicle for propagation of AAV vectors. In the herein disclosed methods of propagation, the rAAV is incubated in avian eggs for longer time periods than in the above-described packaging methods.

In other embodiments, AEF (e.g., CEF) in culture is used as a host vehicle for propagation of AV vectors. The disclosure thus provides AEF as a novel ex vivo method of production of rAAV. Both avian eggs and AEF can be used for large-scale propagation of rAAV, including for clinical endpoints.

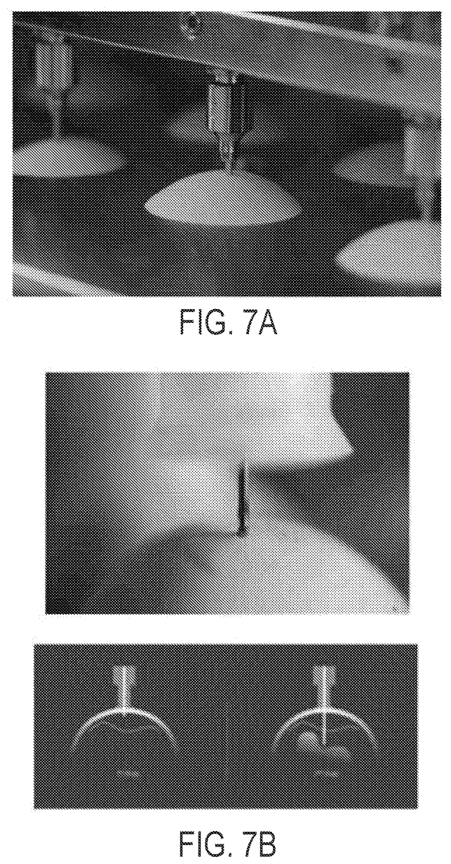

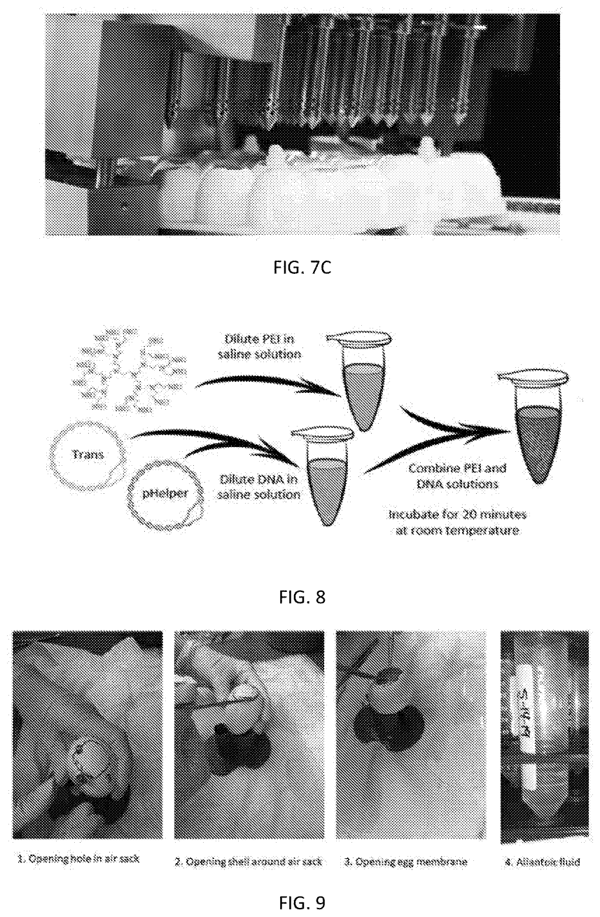

Recombinant AAV virions isolated from a first embryonated egg as above may be propagated in a second embryonated avian egg for larger scale growth. The first embryonated avian egg (for packaging) and second embryonated avian egg (for propagation) may be isolated from the same “batch” of avian eggs, as handled by an automatic egg handler (or injector). See FIGS. 7A-7C. Alternatively, the first embryonated avian egg may be isolated from a different batch of eggs than the second embryonated avian egg.

The methods may subsequently comprise inoculating an embryonated avian egg or avian embryonic fibroblast (AEF) cells with the purified rAAV, and propagating the rAAV by incubating the avian egg (e.g., chicken egg) or AEF (e.g., chicken embryonic fibroblast (CEF), and isolating the rAAV from the egg or AEF. In particular embodiments, the chorioallantoic membrane (CAM) of the egg is inoculated. In other embodiments, the allantoic cavity of the egg is inoculated.

In some embodiments, the same layer or membrane of the egg in which transfection was performed to package the vectors is used for this inoculating step for propagation. In other embodiments, a different layer or membrane of the egg is used for this inoculating step.

Exemplary automatic egg injectors suitable for use in the disclosed methods of propagating AAV in avian eggs include, but are not limited to, injectors manufactured by Sanovax. These injectors have demonstrated suitability for use in growth of influenza vaccine in embryonated chicken eggs.

The egg may be incubated for about 5 days, about 6 days, about 7 days, about 8 days, about 9 days, about 10 days, about 11 days, about 12 days, about 13 days, or about 15 days. In some embodiments, the egg is incubated for about 7 days.

The production methods may subsequently include the step(s) of further purifying and/or concentrating the purified rAAV by tangential flow filtration and/or centrifugation, thereby producing further purified and/or concentrated rAAV. The rAAV particles may be further purified and/or concentrated using any method known in the art, e.g., by tangential flow filtration (TFF), dialysis membrane filtration, and/or centrifugation (e.g., using centrifugation filtration devices, e.g., at 150 kD Molecular weight cut-off (MWCO) membrane filter devices, see, e.g., products from Orbital Bioscience and Millipore). Exemplary commercially available TFF systems and cartridges include products from GE Healthcare Life Sciences (see, e.g., the Midgee products) 5 and Pall Corporation (see, e.g., Minimate™ TFF System).

In particular embodiments, the egg is incubated for about 7-10 days. After about 7-10 days, the rAAV particles in the CAM may be propagated to a suitable level. At this time point, the mean surface area of the CAM is about 65 square centimeters, which is approximately the same size as a 100 mm petri dish.

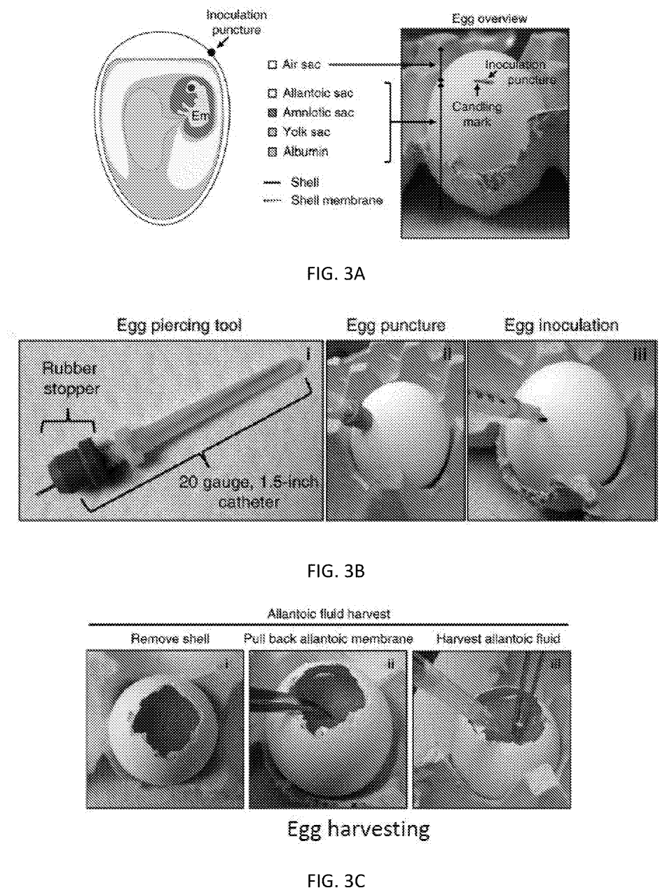

In certain embodiments, the embryonated egg is candled prior to inoculation and/or prior to harvesting. As used herein, “candling” refers to the process of holding the egg, or parts of the egg, in front of a light source, such as a candle, light bulb or fluorescent light source, to determine the stage of development of the embryo (see FIG. 3A). Candling may reveal whether the embryo is alive or viable.

In some embodiments, the rAAV is further propagated in mammalian cells or insect cells. The mammalian cells may be, for example, HEK293 cells, baby hamster kidney (BHK) cells, or HeLa cells. The insect cells may be, for example, Sf9 cells.

In some embodiments, the egg inoculated with rAAV is incubated in a shaker, a spinner or an automatic eggs incubator. In some embodiments, the inoculated AEF cultures are incubated in a shaker flask, a spinner flask, a cellbag, or a bioreactor. In other embodiments, inoculated mammalian cells and/or insect cells are incubated in a shaker flask, a spinner flask, a cellbag, or a bioreactor.

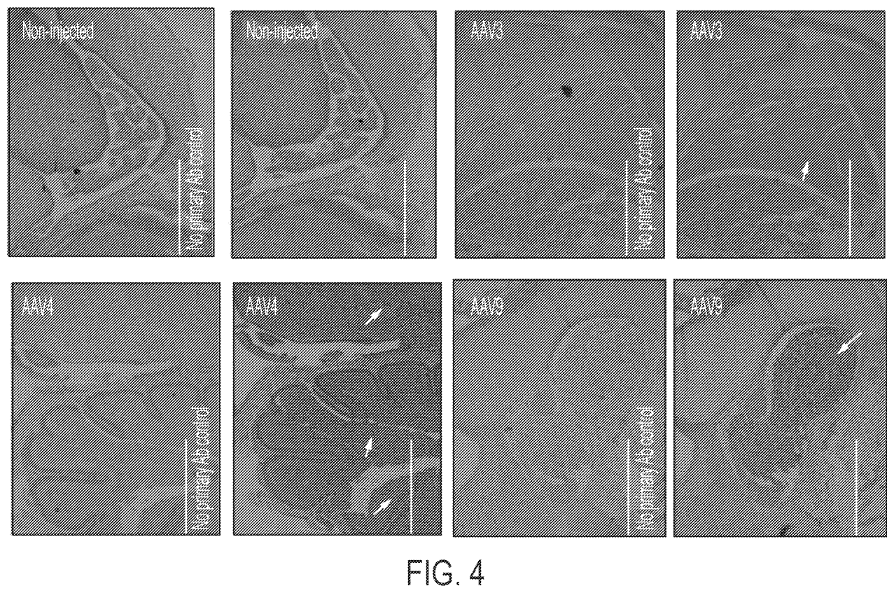

In some aspects, the disclosed production methods comprise propagating rAAV particles in embryonated avian eggs wherein the rAAV may have been packaged in a vehicle other than avian eggs, such as baculovirus. In these methods, avian eggs (e.g., chicken eggs) are used as a host vehicle for propagation of AAV vectors (see FIG. 4).

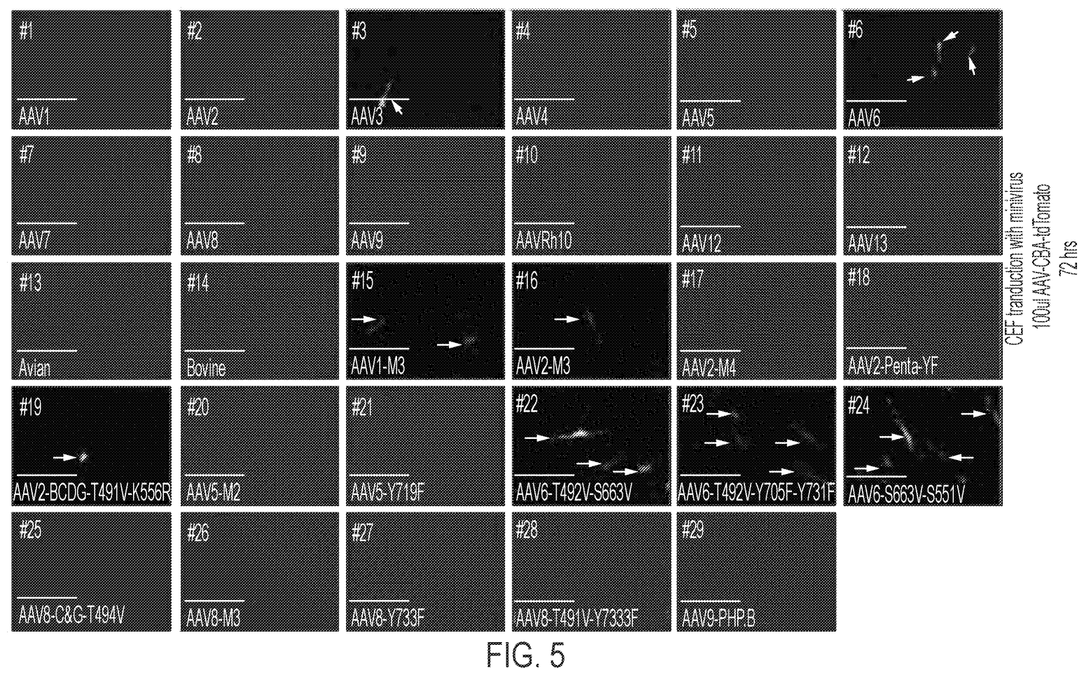

In other aspects, the disclosed production methods comprise propagating rAAV particles in AEF wherein the rAAV may have been packaged in a vehicle other than avian eggs, such as baculovirus. In particular embodiments, the rAAV particles are propagated in CEF (see FIG. 5).

Any of the disclosed methods may be used to procure the successful isolation of purified and/or concentrated rAAV at a titer of at least 1×10⁸, at least 5×10⁸, at least 1×10⁹, at least 5×10⁹, at least 1×10¹⁰, at least 2×10¹⁰, at least 3×10¹⁰, at least 4×10¹⁰, at least 5×10¹⁰, at least 1×10¹¹ vector, at least 2×10¹¹ vector, at least 5×10¹¹, at least 1×10¹², at least 5×10¹², at least 1×10¹³, at least 5×10¹³, at least 1×10¹⁴, at least 5×10¹⁴, at least 1×10¹⁵, or at least 5×10¹⁵ vector genomes (vg)/ml. In particular, the disclosed methods may procure the isolation of purified and/or concentrated rAAV from a single egg or pooled eggs at a titer of at least about 5×10¹⁰ vector genomes (vg)/ml. In particular, the disclosed methods may procure the isolation of purified and/or concentrated rAAV from a single egg at a titer of at least about 5×10¹⁰ vector genomes (vg)/ml.

These titers may be recovered after the injection and harvesting of AAV vectors having an AAV1, an AAV2, an AAV3, an AAV4, an AAVS, an AAV6, an AAV7, an AAV8, an AAV9, an AAV10, an AAV1-M3, an AAV2-M3, an AAV2 (tripYF), an AAV2(quadYF), an AAV2(pentaYF), an AAV2-BCDG(T491V+K556R), an AAVS-M2, an AAVS(Y719F), an AAV6, an AAV6(T492V+S663V), an AAV6(T492V+Y705F+Y731F), an AAV6(S551V+S663V), an AAV8-C&G(T494V), an AAV8-M3, an AAV8(Y733F), an AAV8(T494V+Y733F), an AAV8(Y275F+Y447F+Y733F) or an AAV9-PHP.B serotype. In various embodiments, these titers are recovered after injection and harvesting of AAV vectors having a serotype of a capsid variant with an amino acid substitution in a tyrosine residue. In particular embodiments, these titers are recovered after injection and harvesting of AAV vectors having an AAV6 or an AAV8(Y275F+Y447F+Y733F) serotype.

AAV vector genome concentrations (or titers) may be measured by any method known in the art. Exemplary methods include quantifiable polymerase chain reaction (qPCR). Following purification steps, an amount of about 200 μl of pure AAV vector per egg may be recovered. In other embodiments, amounts of about 50 μl, 75 μl, 100 μl, 150 μl, 175 μl, 180 μl, 190 μl, 210 μl, 220 μl, 225 μl, 250 μl, or 300 μl of pure AAV vector per egg may be recovered.

Advantageously, the methods, compositions and expression vehicles of the present disclosure are adaptable to the existing machinery and methods of the vaccine production industry. Vaccine production in embryonated chicken eggs has been approved by the FDA. The 2015 estimated global capacity for influenza virus production in chicken eggs was 6.4 billion doses (1).

Accordingly, provided herein are methods of improving rAAV production that are adapted to largescale manufacturing and/or are compatible with GLP/GMP guidelines.

In some embodiments of the disclosed packaging methods, the nucleic acid comprising the transgene comprises a transgene flanked by inverted terminal repeats (ITRs). Accordingly, the recovered rAAV of the disclosure comprise a transgene flanked by ITRs. The AAV ITRs may be of an AAV2 serotype.

Further provided herein are expression vehicles for packaging and producing rAAV comprising embryonated avian eggs (e.g., chicken eggs).

In some embodiments, the egg is adapted for production of isolated rAAV at a titer of a titer of at least 1×10⁸, at least 5×10⁸, at least 1×10⁹, at least 5×10⁹, at least 1×10¹⁰, at least 5×10¹⁰, at least 1×10¹¹, at least 2×10¹¹ vector, or at least 5×10¹¹ vector genomes (vg)/ml. The egg may be adapted for production of isolated rAAV at a titer of at least 5×10¹⁰ vector genomes (vg)/ml.

The avian egg may comprise and stably express rAAV that is of an AAV1, an AAV2, an AAV3, an AAV4, an AAV5, an AAV6, an AAV7, an AAV8, an AAV9, an AAVrh10, an AAV12, or an AAV13 serotype. Alternatively, the avian egg may comprise and stably express rAAV that is of an AAV1-M3, an AAV2-M3, an AAV2(tripYF), an AAV2(quadYF), an AAV2(pentaYF), an AAV2-BCDG(T491V+K556R), an AAV5-M2, an AAVS(Y719F), an AAV6(T492V+S663V), an AAV6(T492V+Y705F+Y731F), an AAV6(S551V+S663V), an AAV8-C&G(T494V), an AAV8-M3, an AAV8(Y733F), an AAV8(T494V+Y733F), an AAV8(Y275F+Y447F+Y733F) or an AAV9-PHP.B serotype; or the serotype of another capsid variant.

In some embodiments, a transgene encodes an enzyme, hormone, antibody, receptor, ligand, or other protein. In some embodiments, a transgene encodes a therapeutically useful protein. In some embodiments, a transgene encodes a reporter protein (e.g., EGFP or tdTomato). In some embodiments, a transgene encodes an RNA, for example a regulatory RNA such as a siRNA or other regulatory RNA (e.g., an RNA that can be therapeutically useful).

In some embodiments, a transgene (e.g., encoding a therapeutic agent) is operably linked to a promoter. In some embodiments, the therapeutic agent is a polypeptide, a peptide, an antibody or an antigen-binding fragment thereof, a ribozyme, a peptide-nucleic acid, an siRNA, an RNAi, an antisense oligonucleotide, or an antisense polynucleotide.

In some embodiments, a composition comprising an rAAV described herein can be used to treat a mammalian subject (e.g., a human). In some embodiments, the subject has cancer, diabetes, autoimmune disease, kidney disease, cardiovascular disease, pancreatic disease, intestinal disease, liver disease, neurological disease, neuromuscular disease, Bratten's disease, Alzheimer's disease, Huntington disease, Parkinson's disease, pulmonary disease, an ai-antitrypsin deficiency, neurological disability, neuromotor deficit, neuroskeletal impairment, ischemia, stroke, a lysosomal storage disease, Pompe disease, Duchenne Muscular Dystrophy, Friedreich's Ataxia, Canavan disease, Aromatic L-amino acid decarboxylase deficiency, Hemophilia A/B, or other disease, or any combination thereof.

In some embodiments, the AAV6 (serotype 6) rep and cap genes are used for the second rHSV virus. However, one or more rep and/or cap genes form other AAV serotypes can be used (e.g., from AAV1, AAV2, AAV3, AAV4, AAV5, AAV7, AAV8, AAV9, AAV10, AAV11, AAV12, AAV13 or other serotype). In some embodiments, one or more variant cap genes can be used. In some embodiments, the rep and/or cap genes can be under the control of their natural AAV promoter. However, in some embodiments, they can be under the control of one or more constitutive or inducible promoters.

It should be appreciated that any cell or cell line that is known in the art to propagate rAAV particles can be used in embodiments of the methods disclosed herein, following the packaging of rAAV in embryonated chicken eggs. In some embodiments, a cell used to propagate rAAV in the methods disclosed herein is a mammalian cell. Non-limiting examples of mammalian cells that can be used to propagate rAAV are HEK293 cells, COS cells, HeLa cells, HeLaS3, BHK cells, CHO cells or PER.C6® (see, e.g., ATCC® CRL-1573™, ATCC® CRL-1651™, ATCC® CRL-1650™, ATCC® CCL-2, ATCC® CCL-2.2, ATCC® CCL-10™, or ATCC® CCL-61™). In some embodiments, a cell used to propagate rAAV particles is an insect cell. An example of insect cells includes Sf9 cells (see, e.g., ATCC® CRL-1711™). The Sf9 cells may be Sf9 producer cells that comprise rep and/or cap genes from one or more AAV serotypes or pseudotypes for producing rAAV particles (see, e.g., Mietzsch et al. OneBac: platform for scalable and high-titer production of adeno-associated virus serotype 1-12 vectors for gene therapy. Hum Gene Ther. 2014 Mar.; 25(3):212-22; and Aslanidi et al. An inducible system for highly efficient production of recombinant adeno-associated virus (rAAV) vectors in insect Sf9 cells. Proc Natl Acad Sci USA 2009 106: 5059-5064).

A cell lysate comprising propagated rAAV may be produced using any method known in the art, e.g., by microfluidization, sonication, freeze/thawing, or hypotonic lysis of cells comprising the rAAV particles. The cells comprising the rAAV particles may be in pellet form, either frozen or thawed. In some embodiments, insect cell lysate is produced by microfluidization of insect cells comprising the rAAV particles. In some embodiments, mammalian cell lysate is produced by hypotonic lysis of mammalian cells comprising the rAAV particles. Purifying the rAAV particles from the supernatant using cation exchange chromatography in a method described herein may be accomplished using any method known in the art, e.g., sulfopropyl (SP) column chromatography or carboxymethyl (CM) chromatography (see, e.g., HiPrep™ Sp Fast Flow 16/10, HiPrep™ CM FF 16/10, and HiPrep™ SP XL 16/10 available from GE Healthcare). In some embodiments, the cation exchange chromatography comprises applying the supernatant to a column, washing with a low pH (e.g., pH between 2-5, 3-5, or 3-4) buffer (e.g., sodium citrate buffer), and eluting in a higher pH (e.g., pH 5-8, 5-7, 5-6 or 6-7) buffer (e.g., sodium citrate buffer). Reference is made to US Patent Publication No. 2017/0130208, published on May 11, 2017, incorporated herein by reference.

In some embodiments of methods described herein, the methods may further comprise measuring the purity and/or the amount of intact rAAV particles in the purified rAAV particles. Measuring the purity and/or the amount of intact rAAV particles in the purified rAAV particles may be accomplished using any method known in the art. In some embodiments, measuring the purity and/or the amount of intact 25 rAAV particles comprises an immunoassay, a nucleic acid hybridization-based assay (e.g., a dot-blot assay), SDS-PAGE followed by either Coomassie blue or silver staining, visualization with an electron microscope, a PCR assay, an infectious center assay (e.g., green fluorescent cell assay), or combinations thereof. In some embodiments, measuring the purity and/or the amount of intact rAAV particles comprises an immunoassay that includes antibodies specific for intact capsids (e.g., an antibody described herein or an anti-AAV2-clone A20 or anti-AAV1-clone ADK1a, both available from PROGEN Biotechnik GmbH, Catalog number 61055 and 610150, Heidelberg, Germany, or anti-AAV-8-clone ADK8 or anti-AAV9-clone ADK9, both available from American Research Products, Inc. Catalog number 03-651160 and 03-651162, Waltham, Mass.), antibodies for denatured capsids (e.g., Anti-AAV, VP1/VP2/VP3 clone B1 available from American Research Products, Inc. Catalog number 03-61058, Waltham, Mass.; Wistuba A, et al. (1995), J. Virol, 69: 5311-5319), and/or antibodies specific for VP1u externalization (e.g., Anti-AAV, VP1 clone A1 available from American Research Products, Inc. Catalog number 03-10 61056; Wistuba, A. et al. (1997) J. Virol. 71: 1341-1352; Wobus, C E. et al. (2000) J. Virol 74 (19): 9281-9292).

rAAV Particles and Methods of Producing rAAV Particles

Aspects of the disclosure relate to recombinant AAV (rAAV) particles and methods of purifying the rAAV particles. The purified rAAV particles have many uses, e.g., in methods and pharmaceutical compositions for treating a disease in a subject in need thereof (e.g., a subject having a disease involving reduced protein expression that may be treated with gene therapy), in rAAV particle-derived vaccines, for infecting cells to screen rAAV particles for a desired phenotype (e.g., upregulation of a protein or polypeptide of interest in the cell), or for infecting animals to screen for pharmacokinetics and/or therapeutic efficacy of an rAAV.

In some embodiments, recombinant rAAV particles comprise a nucleic acid vector. In some embodiments, the nucleic acid vector comprises (a) one or more heterologous nucleic acid regions comprising a transgene encoding a protein or polypeptide of interest or encoding an RNA of interest (e.g., a microRNA or a small hairpin RNA) and (b) one or more regions comprising inverted terminal repeat (ITR) sequences (e.g., wild-type ITR sequences or engineered ITR sequences) flanking the one or more heterologous nucleic acid regions. In some embodiments, the nucleic acid vector is encapsidated by a viral capsid. In some embodiments, the nucleic acid vector is circular. In some embodiments, the nucleic acid vector is single-stranded. In some embodiments, the nucleic acid vector is double-stranded. In some embodiments, a double-stranded nucleic acid vector may be, for example, a self-complimentary vector that contains a region of the nucleic acid vector that is complementary to another region of the nucleic acid vector, initiating the formation of the double-strandedness of the nucleic acid vector.

Accordingly, in some embodiments, a rAAV particle comprises a viral capsid and a nucleic acid vector as described herein, which is encapsidated by the viral capsid. In some embodiments, the viral capsid comprises 60 capsid protein subunits comprising VP1, VP2 and VP3. In some embodiments, the VP1, VP2, and VP3 subunits are present in the capsid at a ratio of approximately 1:1:10, respectively. In some embodiments, the nucleic acid vector comprises (1) one or more heterologous nucleic acid regions comprising a transgene encoding a protein or polypeptide of interest, (2) one or more nucleic acid regions comprising a sequence that facilitates expression of the heterologous nucleic acid region (e.g., a promoter and/or enhancer), and (3) one or more nucleic acid regions comprising a sequence that facilitate integration of the heterologous nucleic acid region (optionally with the one or more nucleic acid regions comprising a sequence that facilitates expression) into the genome of the subject. In some embodiments, viral sequences that facilitate integration comprise Inverted Terminal Repeat (ITR) sequences. In some embodiments, the nucleic acid vector comprises one or more heterologous nucleic acid regions comprising a transgene encoding a protein or polypeptide of interest or an RNA of interest operably linked to a promoter, wherein the one or more heterologous nucleic acid regions are flanked on each side with an ITR sequence. The ITR sequences can be derived from any AAV serotype (e.g., 1, 2, 3, 4, 5, 6, 7, 8, 9, 10) or can be derived from more than one serotype. In some embodiments, the ITR sequences are derived from AAV2. ITR sequences and plasmids containing ITR sequences are known in the art and commercially available (see, e.g., products and services available from Vector Biolabs, Philadelphia, Pa.; Cellbiolabs, San Diego, Calif.; Agilent Technologies, Santa Clara, Ca; and Addgene, Cambridge, Mass.; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci USA. 1996 Nov. 26; 93(24):14082-7; and Curtis A. Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In some embodiments, the nucleic acid vector (e.g., comprising one or more heterologous nucleic acid regions comprising a transgene encoding a protein or polypeptide of interest and optionally the one or more nucleic acid regions comprising a sequence that facilitates expression of the heterologous nucleic acid region) is no more than 6 kilobases, no more than 5 kilobases, no more than 4 kilobases, or no more than 3 kilobases in size. In some embodiments, the nucleic acid vector (e.g., comprising one or more heterologous nucleic acid regions comprising a transgene encoding a protein or polypeptide of interest and optionally the one or more nucleic acid regions comprising a sequence that facilitates expression of the heterologous nucleic acid region) is between 4 and 6 kilobases in size, e.g., 4-6 kilobases, 4-5 kilobases, or 4.2-4.7 kilobases.

Methods of producing rAAV particles and nucleic acid vectors are also known in the art and commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, the nucleic acid vector may be combined with one or more plasmids, e.g., that contain a rep gene (e.g., encoding Rep78, Rep68, Rep52 and Rep40) and a cap gene (encoding VP1, VP2, and VP3), and transfected into a producer cell line such that the rAAV particle can be packaged and subsequently purified.

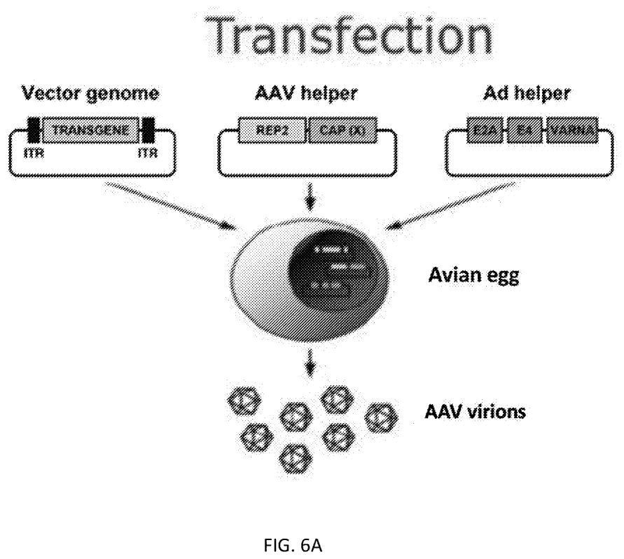

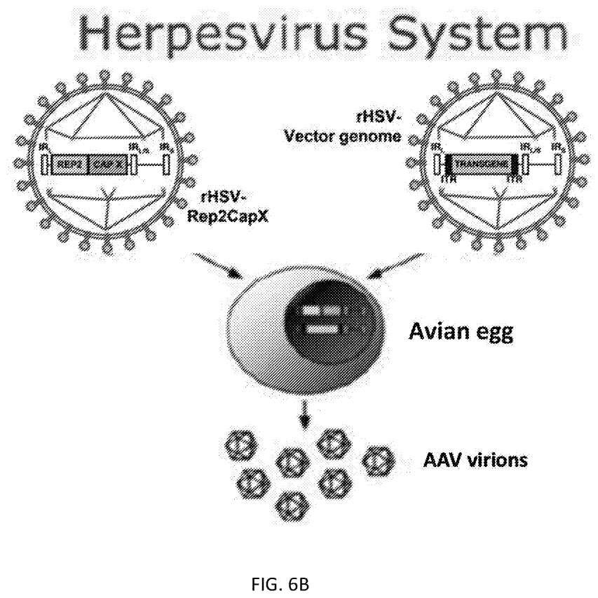

In some embodiments, the one or more helper plasmids comprise a first plasmid comprising a rep gene and a cap gene and a second Ad helper plasmid comprising a E1a gene, a E1b gene, a E4 gene, a E2a gene, and a VA RNA (or “VA”) gene (see FIGS. 6A and 12). Collectively, the Ela, E1b, E4, E2a and VA genes may be referred to herein as “adenovirus helper genes.” In some embodiments, the rep gene is a rep gene derived from AAV6 and the cap gene is derived from AAV6. In some embodiments, the rep gene is a rep gene derived from AAV2 and the cap gene is derived from AAV2. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, Pa.; Cellbiolabs, San Diego, Calif.; Agilent Technologies, Santa Clara, Ca; and Addgene, Cambridge, Mass.; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adenoassociated viral vector reference standards, Molecular Therapy, Vol. 16, 1185-1188).

In some aspects, provided herein is a method for large-scale propagation of rAAV comprising the use of chorioallantoic and/or allantoic fluid of embryonated eggs that is cultured, infected or incubated to produce rAAV particles in a shaker flask, a spinner flask, a cellbag, or a bioreactor (e.g., a Wave reactor).

In some embodiments of the disclosed methods, the AAV capsids can be derived from any AAV serotype. In some embodiments, AAV capsids are derived from AAV1, AAV2, AAV3, AAV4, AAV5, AAV6, AAV7, AAV8, AAV9, AAV10, AAV11, AAV12, AAV13 or other AAV serotypes (e.g., a hybrid serotype harboring sequences from more than one serotype). In particular embodiments, the AAV capsids are derived from AAV1, AAV2, AAV9, AAVS, AAV3, AAV4, AAV6 or AAV8 serotypes.

In other embodiments, the AAV capsid may be a capsid variant. In particular, the AAV ITRs may be of an AAV1-M3, an AAV2-M3, an AAV2(tripYF), an AAV2(quadYF), an AAV2(pentaYF), an AAV2-BCDG(T491V+K556R), an AAVS-M2, an AAVS(Y719F), an AAV6(T492V+S663V), an AAV6(T492V+Y705F+Y731F), an AAV6(S551V+S663V), an AAV8-C&G(T494V), an AAV8-M3, an AAV8(Y733F), an AAV8(T494V+Y733F), an AAV8(Y275F+Y447F+Y733F) or an AAV9-PHP.B serotype; or the serotype of another capsid variant. Any capsid variant having an amino acid substitution in a tyrosine residue is particularly suitable for use with the methods of the present disclosure.

Transfection or Infection of Cells to Introduce AAV Rep and Cap Proteins and/or One or More Genes of Interest

There are numerous methods by which AAV rep and cap genes, AAV helper genes, and one or more genes of interest can be introduced into cells to produce or package rAAV. In some embodiments of any one of the rAAV production methods disclosed herein, AAV rep and cap genes and one or more genes of interest can be introduced into cells by transfection of one or more plasmid vectors harboring AAV rep and cap genes and one or more genes of interest. In some embodiments of any one of the rAAV production methods disclosed herein, AAV rep and cap genes and one or more genes of interest can be introduced into cells by infection of viral vectors harboring AAV rep and cap genes and one or more genes of interest can be introduced into cells.

AAV rep and cap genes may be harbored by one or even more than one (e.g., two or three) vectors (e.g., plasmids or viral vectors). Similarly, more than one (e.g., two or three) genes of interest (e.g., a gene encoding a therapeutic protein) may be harbored by one or even more than one (e.g., two or three) vectors (e.g., plasmids or viral vectors).

One or more helper plasmids are produced or obtained, which comprise rep and cap ORFs/genes for the desired AAV serotype or pseudotype (e.g., a rep2cap9 plasmid or a rep2cap2 plasmid) and the adenoviral VA, E2A (DBP), and E4 genes under the transcriptional control of their native promoters (e.g., a pXX6 plasmid). (See FIGS. 6A, 6B and 12.)

Plasmids carrying certain rep and cap and other genes needed for rAAV packaging, such as the E1a gene, E1b gene, E4 gene, E2a gene, and VA (VA RNA) gene, are also referred to as helper plasmids. Some non-limiting examples of helper plasmid vectors that are commercially available are pDF6, pRep, pDM, pDG, pDP1rs, pDP2rs, pDP3rs, pDP4rs, pDP5rs, pDP6rs, pDG(R484E/R585E), and pDP8.ape plasmids. Each of the pDPXrs helper plasmids, where X indicates the serotype of the capsid (or capX) gene contained in the helper plasmid, encode a red fluorescent protein (RFP). As such the levels of rAAV genome, which indicates level of packaged AAV particles, may be evaluated using anti-RFP immunohistochemistry (histology).

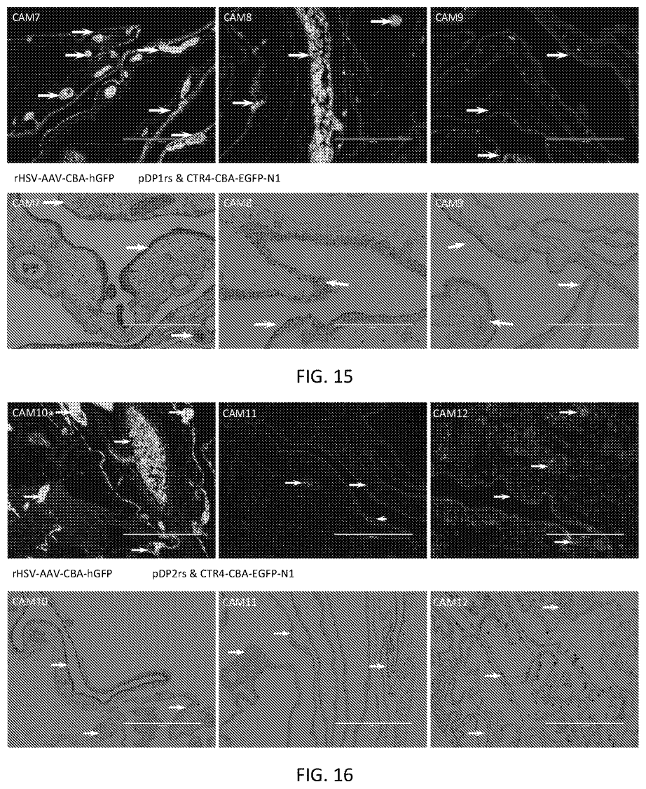

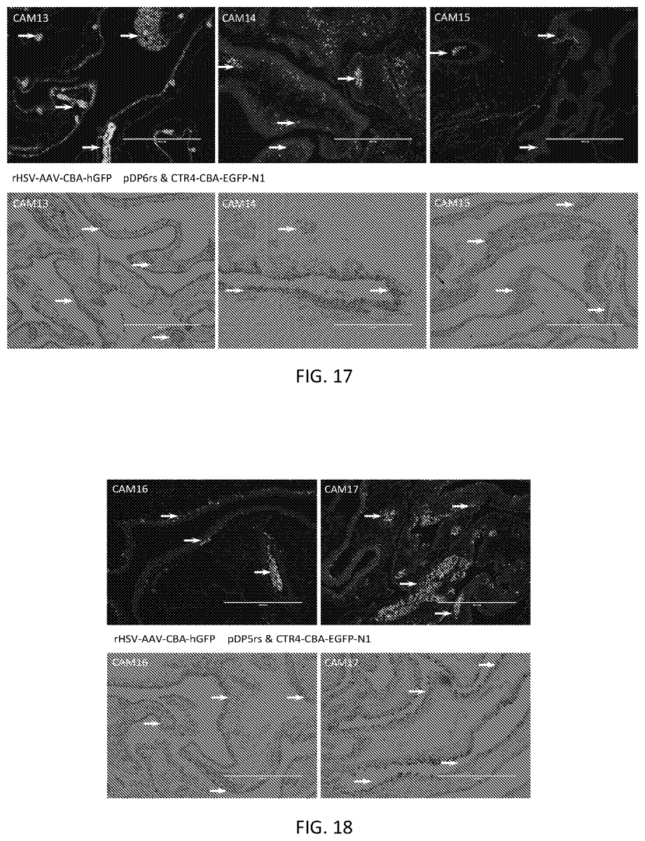

In some embodiments, a pDP1rs helper plasmid is used in rAAV packaging (see FIG. 15). In some embodiments, a pDP2rs helper plasmid is used in rAAV packaging (see FIG. 16). In some embodiments, a pDP3rs helper plasmid is used in rAAV packaging. In some embodiments, a pDP4rs helper plasmid is used in rAAV packaging. In some embodiments, a pDP5rs helper plasmid is used in rAAV packaging (see FIG. 18). In some embodiments, a pDP6rs helper plasmid is used in rAAV packaging (see FIG. 17).

In some embodiments, the disclosed methods comprise producing recombinant AAV (rAAV) by inoculating an embryonated avian egg with a first recombinant CELO (rCELO) viral particle comprising a transgene and a second rCELO viral particle comprising AAV rep and cap genes. In such methods, an rCELO virus may supply the E1a gene and/or other AAV helper genes. Use of rCELO may be used to generate rAAV of non-avian origin, in accordance with the disclosed methods.

Molecular biology techniques to develop plasmid or viral vectors (eg., rHSV or baculovirus vectors) with either AAV rep and cap genes or one or more genes of interest are commonly known in the art.

It is to be understood that any combination of vectors can be used to introduce AAV rep and cap genes and one or more genes of interest to a cell in which rAAV particles are to be produced or packaged. For example, a first rHSV encoding a transgene flanked by AAV inverted terminal repeats (ITRs), and a second rHSV encoding AAV rep and cap genes can be used (see below section). In some embodiments, a combination of transfection and infection is used by using both plasmid vectors as well as viral particles. In some embodiments, one or more helper genes is constitutively expressed by the cells and does not need to be transfected on infected into the cells.

An AAV rep gene encodes rep proteins Rep78, Rep68, Rep52 and Rep40. An AAV cap gene encodes capsid proteins VP1, VP2 and VP3 of a specific capsid serotype.

In some embodiments, the rep and/or cap genes are operably linked to a promoter. A promoter may be constitutive, inducible or synthetic. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Non-limiting examples of constitutive mammalian promoters include various housekeeping gene promoters, as exemplified by the β-actin promoter (e.g., chicken β-actin promoter) and human elongation factor-1 α (EF-1α) promoter. Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline. Synthetic promoters are also contemplated herein. A synthetic promoter may comprise, for example, regions of known promoters, regulatory elements, transcription factor binding sites, enhancer elements, repressor elements, and the like.

The rHSV Transduction Protocol: A Method for Producing rAAV Using rHSV Infection

In some embodiments, the packaging vector comprises a recombinant herpes simplex virus vector or particle (an rHSV vector or rHSV particle). The use of rHSV to package rAAV virions should reduce the cost of production relative to the PEI transfection by eliminating the use of plasmids and transfection reagents.

HSV-1 can fully support AAV replication and packaging (Knipe, 1989; Advances in Virus Research 37:85-123, Buller, J Virol. 1981 October; 40(1):241-7, Mishra and Rose, Virology. 1990 December; 179(2):632-9, Weindler et al., J Virol. 1991 May; 65(5):2476-83) in other production formats and was found to be effective in embryonated avian eggs. In some embodiments, certain HSV-1 genes required to replicate and package non-avian AAV (e.g., UL5, UL8, UL52 and UL29—Weindler et al., J Virol. 1991 May; 65(5):2476-83) are maintained within one or more rHSV used for co-infection. These genes encode components of the HSV-1 core replication machinery and by themselves form nuclear prereplication centers that develop into mature replication foci (Weindler et al., J Virol. 1991 May; 65(5):2476-83, Knipe, D. M., Advances in Virus Research 37:85-123). In the context of egg inoculation, recombinant HSV-1 viruses are also used to supply the helper functions needed for rAAV production.

In various embodiments, the rHSV particle is of mammalian origin (e.g., primate, for example human origin). In some embodiments, two different rHSV viruses are used, each containing a different gene cassette. See FIG. 6B. In addition to supplying the necessary helper functions, each of these rHSV viral particles is engineered to deliver different AAV (and other) genes to the embryonated eggs upon inoculation. In some embodiments, a first recombinant HSV (e.g., rHSV1 in FIG. 6B) contains a transgene nucleic acid, for example having sequences encoding a transgene, along with promoter elements necessary for expression of the gene. Generally, the transgene nucleic acid is inserted between two AAV inverted terminal repeats (ITRs). In some embodiments, a second rHSV contains a gene cassette in which the rep and cap genes from AAV are inserted into the HSV genome. The rep genes are responsible for replication of the rAAV genome in host cells infected with AAV. The cap genes encode proteins that comprise the capsid of the rAAV produced by the infected cells.

In some embodiments, packaging vectors or viral particles comprising helper genes but that do not also encode AAV rep or cap genes may be used.

In some embodiments, the first and second rHSV viruses are used to co-inoculate or co-infect (e.g., simultaneously or at approximately the same time) the embryonated eggs (e.g., the embryonated avian eggs). In some embodiments, two or more rHSV viruses that are used for a simultaneous co-infection protocol to produce rAAV can be produced from HSV-1. In some embodiments, HSV used to produce rAAV can be of any variant (e.g., HSV-1 or HSV-2 or any other serotype or variant or mutant forms thereof).

In some embodiments, the first and second rHSV viruses can be produced using similar techniques (e.g., by homologous recombination into the HSV-1 tk gene).

In some embodiments, one or more of the AAV rep or cap genes are introduced or delivered to eggs using more than one rHSV particles. For example, two rHSVs, one that harbors rep and cap genes and another that harbors a transgene, may be used.

In some embodiments, the one or more rHSV vectors comprise a first rHSV encoding a gene of interest flanked by AAV ITRs, and, a second rHSV encoding AAV rep and cap genes. In some embodiments, the AAV ITRs are AAV2 ITRs. In some embodiments, the AAV ITRs are from AAV1, AAV2, AAV3, AAV4, AAV5, AAV6, AAV7, AAV8, AAV9, AAV10, AAV11, AAV12, AAV13 or other AAV serotype.

In some embodiments of the disclosed transduction protocol methods, in place of an rHSV particle, a mammalian adenovirus particle or an avian adenovirus particle (a CELO) may be used to supply the AAV rep and cap genes. In some embodiments, a mammalian adenovirus or an avian adenovirus particle (a CELO) may be used to supply the AAV rep and cap genes and/or other helper genes. Accordingly, in some embodiments, the packaging vectors (or particles) comprise a mammalian adenovirus. In some embodiments, an adenovirus serves as the transgene vector, and accordingly comprises a nucleic acid comprising a transgene the disclosed packaging methods. In some embodiments, the first packaging vector that comprises a nucleic acid comprising a transgene, and the second packaging vector that comprises a nucleic acid encoding AAV rep and cap genes, are mammalian adenoviruses.

In some embodiments, the packaging vectors (or particles) comprise a CELO virus. In some embodiments, a CELO serves as the transgene vector, and accordingly supplies the transgene to the disclosed packaging methods. In some embodiments, the first packaging vector that comprises a nucleic acid comprising a transgene, and the second packaging vector that comprises a nucleic acid encoding AAV rep and cap genes, are CELO adenoviruses.

It should be appreciated that AAV rep, cap and helper genes (e.g., E1a gene, E1b gene, E4 gene, E2a gene, or VA gene) can be derived from any AAV serotype.

In some embodiments, the AAV cap gene is an AAV9, AAV2 or AAV5 cap gene. In other embodiments, the AAV cap gene is derived from AAV1, AAV3, AAV4, AAV6, AAV7, AAV8, AAV10, AAV11, AAV12, AAV13 or other AAV serotype (e.g., a hybrid serotype harboring sequences from more than one serotype). In some embodiments, rep and cap genes for the production of a rAAV particle is from different serotypes. For example, the rep gene can be from AAV8 whereas the cap gene can be from AAV6.

In some embodiments, the rep and/or cap genes are operably linked to a promoter. In some embodiments, the transgene is operably linked to a promoter. In some embodiments, the rep gene, cap gene, and/or transgene may be operably linked to a natural (e.g., homologous) promoter, or alternatively a heterologous promoter. In some embodiments, a promoter may be constitutive, inducible or synthetic.

In some embodiments, the rep gene, cap gene, and/or transgene may be operably linked to other regulatory elements, such as a Woodchuck Hepatitis Virus (WHP) Posttranscriptional Regulatory Element (WPRE).

In some embodiments, a transgene encodes a therapeutic protein. In some embodiments, a therapeutic gene encodes an antibody, a peptibody, a growth factor, a clotting factor, a hormone, a membrane protein, a receptor, a cytokine, a chemokine, an activating or inhibitory peptide acting on cell surface receptors or ion channels, a cell-permeant peptide targeting intracellular processes, a thrombolytic, an enzyme, a bone morphogenetic proteins, a nuclease or other protein used for gene editing, an Fc-fusion protein, an anticoagulant, a nuclease, guide RNA or other nucleic acid or protein for gene editing.

In some embodiments, a therapeutic protein is therapeutic for lysosomal storage disease. In some embodiments, a therapeutic protein is therapeutic for a neurological disability, a neuromotor deficit, a neuroskeletal impairment or a neuromuscular disease.

In some embodiments, a therapeutic protein is therapeutic for a muscular disability or dystrophy, a myopathy or a cardiomyopathy.

### Egg Production Methods

While the methods of the present invention may be carried out on individual eggs, in a commercial setting the method is typically carried out on a plurality of eggs. In general, in a commercial setting, a plurality of eggs are incubated together in a common incubator. A number of automatic egg injection devices have been developed. These include U.S. Pat. No. 5,056,464 to Lewis: U.S. Pat. Nos. 4,903,635 and 4,681,063 to Hebrank; U.S. Pat. No. 5,136,979 to Paul et al.: and U.S. Pat. Nos. 4,040,388, 4,469,047 and 4,593,646 to Miller, each of which are incorporated herein by reference.

The size of the allantoic fluid is related to the stage of embryonic development of the egg to be injected; thus the depth of injection or insertion needed to reach the allantoic fluid will vary depending on the developmental stage of the egg as well as the species and strain of avian egg used. The depth of injection or insertion must be deep enough to place the needle or probe within the allantoic fluid, but not so deep as to pierce the amnion or embryo. Use of a blunt-tip needle helps minimize piercing of the amnion or embryo. In chicken eggs at days 7-15 of embryonation, injection or insertion from ⅛th to ¼th of an inch below the egg shell surface is preferred. Age of embryonation may be determined by candling the egg.

The disclosed methods of rAAV production in embryonated avian eggs embrace incoulation through the uppermost side of the egg; most commercial egg injection apparatus are designed to inject eggs using a needle that is vertical and that travels downward into the egg. See e.g., U.S. Pat. No. 4,469,047 to Miller; U.S. Pat. No. 4,681,063 to Hebrank; U.S. Pat. No. 4,903,635 to Hebrank; U.S. Pat. No. 5,056,464 to Lewis; U.S. Pat. No. 5,136,979 to Paul and Ilich; and published PCT application WO 98/31216 to Bounds, each of which are incorporated herein by reference. The disclosed methods comprise methods of orienting eggs also allow vertical downward injection while avoiding piercing the air cell membrane. Accordingly, a preferred method of injection is downward along the path of the needle since this method of injection is more readily accomplished with minimum modification to existing automatic injection machines.

It will be appreciated by those skilled in the art that the precise location and angle of injection is a matter of choice and could be in any area of the egg's upper surface that overlies the allantoic fluid. Orientation of the needle will depend on the orientation of the egg and the equipment available to carry out the injection. While the orientation of the egg may be about 45 degrees from vertical, the orientation may extend from about 10 degrees up to 180 degrees. Preferred angles of egg orientation include from about 20 degrees to about 45 degrees. Injecting the egg with the long axis of the egg more nearly vertical, i.e., an angle of less than about 10 degrees, increases the chance that the injection needle will traverse the air cell. Where it is desired to avoid piercing the air cell, routine experimentation using eggs of similar age and condition and from the same breed and strain of bird, will allow determination of the minimum angle of egg orientation needed to avoid piercing the air cell in a majority of such eggs.

The automated injection methods of the disclosure may involves delivering rAAV particles or AAV plasmids in fluid form to the interior of an egg using an automated machine which delivers the vaccine to the egg through a needle. The needle can be used to both penetrate the egg shell and deliver the fluid substances, or the opening in the shell can be performed separately in advance of the fluid injection. The egg can be injected at any location within the egg, such as the allantoic fluid or through the CAM.

At about the beginning of the final quarter of incubation, the eggs are transferred from the incubator to a hatcher. This step is known as “transfer”. At transfer, the step of sampling or sensing the allantoic fluid to assess the condition or status of the embryo within the egg may advantageously be carried out, as can injection of beneficial substances into the allantoic fluid. An egg tray may be used in automatic egg injection equipment (see FIG. 7C). The egg tray comprises a base containing a plurality of egg receptacles which are configured to hold the eggs at a desired angle. Typically, eggs are incubated in an incubating tray placed in an incubator or setter machine. Conventional incubating trays include the Chick Master® 54 tray, the Jamesway® 42 tray, and the Jamesway® 84 tray (in each case, the number indicates the number of eggs carried by the tray). The eggs from three Chick Master® 54 trays, or a total of 162 eggs, would be transferred to a single hatcher tray; the eggs from four Jamesway® 42 trays, or a total of 168 eggs, would be transferred to a single hatcher tray; and the eggs from two Jamesway® 84 trays, or a total of 168 eggs, would be transferred to a single hatcher tray. Some incubating trays, such as the La Nationale® incubating tray, which are sufficiently large enough to include a total number of eggs, in this case 132 eggs, such that the eggs from a single incubating tray would be transferred to a corresponding hatcher tray.

Automated machines and methods for simultaneously injecting a large number of eggs are known. In one well known commercial machine, the eggs in the incubating trays are brought under a bank of injectors which house both needles and punches. First, the punches open a hole in the egg shell. Then, the needle is inserted into the egg through the open hole, followed by injection of the fluid. The punch is necessary because the needle is long and thin and cannot repeatedly punch egg shells without bending and/or clogging. This system is described, for example, in U.S. Pat. No. 4,691,063 to Hebrank. In another machine, such as that described in U.S. Pat. No. 6,240,877 B1, the injectors house a single needle which both punches the hole in the egg shell with a closed needle end and then delivers the fluid through a hole in the side of the needle tip.

### Compositions

Aspects of the disclosure relate to compositions comprising rAAV particles or further purified and/or concentrated rAAV particles described herein. In some embodiments, the composition is obtainable by or produced by a method described herein.

In some embodiments, purified rAAV particles or further purified and/or concentrated rAAV particles described herein is/are added to a composition, e.g., a pharmaceutical composition. In some embodiments, the composition comprising rAAV particles has a purity of above 90%, above 91%, above 92%, above 93%, above 94%, above 95%, above 96%, above 97%, above 98%, above 99%, above 99.1%, above 99.2%, above 99.3%, above 99.4%, above 99.5%, above 99.6%, above 99.7%, above 99.8%, above 99.9% or above 99.99%. Purity may be measured using any method known in the art. In some embodiments, purity is measured by determining the amount of capsid proteins, VP1, VP2, and VP3 (which are approximately 87, 72, and 63 kiloDaltons (kD) in size, depending on the serotype) relative to the total protein amount present in the purified rAAV particles or further purified and/or concentrated rAAV particles (e.g., in a pharmaceutical composition). In some embodiments, the amount of capsid proteins is measured using SDS-PAGE followed by a gel stain such as Silver stain or Coomassie Blue stain (e.g., GelCode® Blue reagent from ThermoScientific). The stain may be quantified, e.g., by densitometry or any other method known in the art. In some embodiments, the capsids proteins are present in an amount of at least 90% of the total protein amount (e.g., at least 90% of all bands present in an SDS-PAGE gel as detected by Silver stain or Coomassie stain are capsid protein bands such as at 87, 72, and 63 kiloDaltons). In some embodiments, one or more positive controls are used in a purity measurement or assay, e.g., compositions comprising a known rAAV serotype, optionally at a known concentration.

In some embodiments, the composition comprises a pharmaceutically acceptable carrier. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), pH adjusting agents (such as inorganic and organic acids and bases), sweetening agents, and flavoring agents.

In some embodiments, a composition described herein may be administered to a subject in need thereof. In some embodiments, a method described herein may further comprise administering a composition comprising rAAV particles as described herein to a subject in need thereof. In some embodiments, the subject is a human subject.

In some embodiments, the subject has or is suspected of having a disease that may be treated with gene therapy. Exemplary diseases include, but are not limited to, cystic fibrosis, hemophilia B, San Filippo syndrome, lipoprotein lipase deficiency, alpha-1 antitrypsin deficiency, arthritis, hereditary emphysema, Leber's congenital amaurosis, age-related macular degeneration, muscular dystrophy (duchenne, LGMD2d and 2c), Parkinson's disease, Canavan's disease, Batten's disease, Alzheimer's disease, metachromatic leukodystrophy, alpha-1 antitrypsin deficiency, lipoprotein lipase deficiency, heart failure, rheumatoid arthritis, amyotrophic lateral sclerosis (ALS), spinal muscular atrophy (SMA), ornithine transcarbamylase deficiency, epilepsy, Rett syndrome, lysosomal storage disorders of skeletal muscle or CNS or Pompe disease, or another disease listed in Table 1. These diseases, associated symptoms and signs of the diseases, and methods of diagnosis of the diseases are known in the art and available to the skilled practitioner. Treatment methods involving rAAV particles are also known in the art (see, e.g., Adeno-Associated Virus Vectors in Clinical Trials. Barrie J. Carter. Human Gene Therapy. May 2005, 16(5): 541-550. doi:10.1089/hum.2005.16.541. Published in Volume: 16 Issue 5: May 25, 2005; Neuropharmacology. 2013 June; 69:82-8. doi: 10.1016/j.neuropharm.2012.03.004. Epub 2012 Mar. 17.; Adeno-associated virus (AAV) gene therapy for neurological disease. Weinberg MS1, Samulski R J, McCown T J. Gene therapy for lysosomal storage disorders. Yew N S, Cheng S H. Pediatr Endocrinol Rev. 2013 Nov.; 11 Suppl 1:99-109; Directed evolution of novel adeno-associated viruses for therapeutic gene delivery. Bartel M A, Weinstein J R, Schaffer D V. Gene Ther. 2012 Jun.; 19(6):694-700. doi: 10.1038/gt.2012.20. Epub 2012 Mar. 8; Therapeutic in vivo gene transfer for genetic disease using AAV: progress and challenges. Mingozzi F, High K A. Nat Rev Genet. 2011 May; 12(5):341-55. doi: 10.1038/nrg2988).

The compositions described above may be administered to a subject in any suitable formulation by any suitable method. The route of administration of the composition may be oral, parenteral, by inhalation or topical. The term parenteral as used herein includes intravenous, intraarterial, intraperitoneal, intramuscular, intradermal, intrathoracic, intrathecal, and subcutaneous administration. The compositions described above are typically administered to a subject in an effective amount, that is, an amount capable of producing a desirable result. The desirable result will depend upon the active agent being administered. For example, an effective amount of rAAV particles may be an amount of the particles that are capable of transferring a heterologous nucleic acid to a host organ, tissue, or cell. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

In some embodiments, compositions comprising rAAV particles may be directly introduced into a subject, including by intravenous (IV) injection, intraperitoneal (IP) injection, or in situ injection into target tissue (e.g., muscle). For example, a syringe and needle can be used to inject a rAAV particle composition into a subject. Depending on the desired route of administration, injection can be in situ (e.g., to a particular tissue or location on a tissue), intramuscular, IV, IP, or by another parenteral route. Parenteral administration of rAAV particles by injection can be performed, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the rAAV particles may be in powder form (e.g., lyophilized) for constitution with a suitable vehicle, for example, sterile pyrogen-free water, before use.

To facilitate delivery of the rAAV particles to a subject, the rAAV particles can be mixed with a carrier or excipient. Carriers and excipients that might be used include saline (e.g., sterilized, pyrogen-free saline) saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of rAAV particles to human subjects. Methods for making such formulations are well known and can be found in, for example, Remington: The Science and Practice of Pharmacy, 22nd edition, Pharmaceutical Press, 2012.

In addition to the formulations described previously, the rAAV particles can also be formulated as a depot preparation. Such long acting formulations may be administered by implantation (for example subcutaneously or intramuscularly) or by intramuscular (IM) injection. Thus, for example, the rAAV particles may be formulated with suitable polymeric or hydrophobic materials (for example as an emulsion in an acceptable oil) or ion exchange resins, or as sparingly soluble derivatives.

Further aspects of the disclosure relate to kits, e.g., a kit for performing a method described herein. In some embodiments, the kit comprises water for injection (WFI), sodium citrate and citric acid. In some embodiments, the sodium citrate and citric acid are in a solution (e.g., at molarity described herein), either together (e.g., as a buffer) or separately. In some embodiments, the kit further comprises instructions for use, e.g., instructions for use in a method described herein. In some embodiments, the kit further comprises one or more tubes or other types of containers for cell lysate (e.g., Eppendorf tubes) and/or one or more tubes or other types of containers for waste generated (e.g., 10 mL or 50 mL tubes for collecting flow-through and other wastes that could be produced in a method described herein).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever.

### Examples

### Example 1

PEI Transfection Using a Double Transfection Protocol, for Packaging of rAAV

For the production of a first set of rAAV virions, the double transfection method was used. In a microcentrifuge tube, each of i) 675 ng of a plasmid comprising a nucleic acid encoding an EGFP transgene operably linked to a chicken β-actin (CBA) promoter (CBA-EGFP), ii) 2025 ng of a plasmid encoding AAV nucleic acid (AAV1 rep, AAV1 cap and adenovirus helper genes), iii) 10 μl of 1.5M NaCl, and water were combined (final volume 100 μl). Reagents are shown in Table 1. The solution was then mixed by pipetting several times. The AAV1 rep and cap were derived from human AAV1 virus. In some embodiments, larger volumes of 1.5M NaCl are used, such as 150 μl.

A dilute polyethylenimine (PEI) solution was added into the microcentrifuge containing the plasmids and was mixed by pipetting several times, to prepare the virions for PEI-mediated transfection prior to inoculation. This solution was subsequently incubated at room temperature for 20 minutes, and the plasmids/PEI solution was injected into the CAM of the egg (see FIG. 8). In some embodiments, volumes of PEI of between 10 μl and 500 μl are used for transfection. In some embodiments, volumes of about 250 μl are used (e.g., 244 μl).

### Transfection Using a Triple Transfection Protocol

For the production of a second set of rAAV virions, the triple transfection method was used. In a microcentrifuge tube, i) 675 ng of a plasmid encoding CBA-EGFP, ii) 675 ng of a plasmid encoding AAV1 rep and cap genes, iii) 1350 ng of a plasmid encoding adenovirus helper genes, iv) 10 μl of 1.5M NaCl, and water were combined (final volume 100 μl). The solution was then mixed by pipetting several times. Next, in a second microcentrifuge tube, 30 μl of PEI, 10 μl of 1.5M NaCl, and water were combined to make the final volume 100 μl. Reagents are shown in Table 1. The solution was then mixed by pipetting several times.

The dilute PEI solution was added into the microcentrifuge containing the plasmids and was mixed by pipetting several times. This solution was subsequently at room temperature for 20 minutes, and the plasmids/PEI solution was injected into the CAM of the egg toward the allantoic cavity.

TABLE 1
Reagents for Transfection of Embryonated Chicken Eggs:

| AAV double transfectio | | |
|---|---|---|
| Transgene plasmid | 675 | ng |
| Capsid (AAV) plasmid | 2025 | ng |
| 1.5M NaCl | 10 | μl |
| Water | Up to 100 | μl |
| Total | 100 | μl |

AAV triple transfection

| | | |
|---|---|---|
| Transgene plasmid | 675 | ng |
| Capsid (AAV) plasmid | 675 | ng |
| Adeno helper plasmid | 1350 | ng |
| 1.5M NaCl | 10 | μl |
| Water | Up to 100 | μl |
| Total | 100 | μl |

PEI solution

| | | |
|---|---|---|
| PEI | 30 | μl |
| 1.5M NaCl | 10 | μl |
| Water | 60 | μl |
| Total | 100 | μl |

Allantoic Cavity Inoculation and Harvesting, for Packaging of rAAV Particles

It was sought to package the isolated rAAV in embryonated chicken eggs. It was determined that the optimal conditions for packaging was allantoic cavity inoculation in ten-day old embryos. The eggs were candled to determine the stage of their development (e.g., whether their age had reached ten days). While candling the eggs, a pencil line was drawn around the air sac. Then, using an 18.5 gauge needle, a hole was poked into the air sac. See FIG. 3A.

Next, using a 1.0 mL syringe fitted with a 22.5 gauge needle, the plasmids/PEI mix was injected through the hole into the allantoic cavity while being careful not to stick the embryo.

Finally, the hole was sealed with tape. Any sealant, such as wax, may also be used.

Next, the inoculated eggs were incubated under conditions recommended for viral replication. Eggs were candled at 12-18 hours after inoculation and eggs that were non-viable were discarded.

To harvest the rAAV from the allantoic fluid, eggs were refrigerated for at least 2 hours post-incubation. Once the embryo was no longer viable, the egg was opened by tapping on the shell just above the air sac until the shell broke.

Then, sterile scissors were used to cut away the shell around the air sac. The allantoic fluid was aspirated with a syringe or a pipette. Typically, 10 mL was harvested from each egg. See FIGS. 3A-3C and 9.

At 72 hours after transfection, allantoic fluid was harvested and clarified by centrifugation at 350,000 g per hour. See FIG. 10. The AAV particles were subsequently isolated using a discontinuous Iodixanol gradient. Samples were buffer exchanged to PBS using an Amicon Ultra filter 100,000 MWCO Centrifugation device (Millipore).

### Example 2. Propagation of rAAV Particles in Embryonated Chicken Eggs, and Validation of Packaging in Mammalian Cells

### Propagation in Chicken Eggs

The rAAV recovered from the above packaging protocol was inoculated into a second batch of embryonated chicken eggs for long-term and larger scale propagation. It was sought to determine whether propagation in eggs could provide higher yields of AAV vector than other methods. Purified rAAV-CBA-EGFP (10 μl) plasmid was injected into ten-day old embryonated chicken eggs and embryos were harvested after 7 days. Embryos were formalin-fixed and brain was harvested to perform histological analysis of EGFP expression (paraffin sections). These results shown in FIG. 4 suggested the feasibility that multiple AAV serotypes could be propagated in embryonated eggs.

Following propagation and purification by iodixanol gradient, an average amount of about 150 μl of pure AAV vector per egg was recovered.

The above experiment was repeated with several different AAV serotypes in an effort to determine the effect of particular serotypes on yield.

Separately, plasmids encoding recombinant AAV expressing tdTomato red fluorescent protein, operably linked to a CBA promoter, were inoculated into chicken embryo fibroblast (CEF). The results shown in FIG. 5 suggested the feasibility that AAV could be packaged in CEF. Several different AAV serotypes were tested in this experiment: an AAV2, an AAV3, an AAV4, an AAVS, an AAV6, an AAV7, an AAV8, an AAV9, an AAV10, an AAV1-M3, an AAV2-M3, an AAV2(tripYF), an AAV2(quadYF), an AAV2(pentaYF), an AAV2-BCDG(T491V+K556R), an AAVS-M2, an AAVS(Y719F), an AAV6, an AAV6(T492V+S663V), an AAV6(T492V+Y705F+Y731F), an AAV6(S551V+S663V), an AAV8-C&G(T494V), an AAV8-M3, an AAV8(Y733F), an AAV8(T494V+Y733F), and an AAV9-PHP.B serotype.

The highest yield of AAV vector recovered from a single egg was after use of vectors having an AAV8(Y275F+Y447F+Y733F) capsid, at a 2.25×10¹¹ vg/ml titer. The second-best yield of AAV vector genomes recovered from a single egg was after use of vectors having a wild-type AAV6 capsid, at a 2.11×10¹¹ vg/ml titer.

### Transfection in Mouse Cells

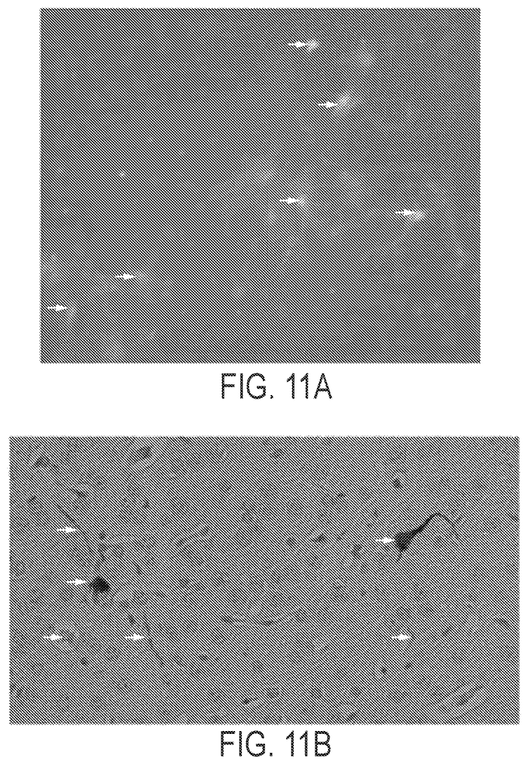

It was determined that purified rAAV1 particles produced in embryonated chicken eggs can successfully transduce mammalian cells. Recombinant AAV1-CBA-EGFP virus was produced in embryonated eggs at a titer of 4.47×10¹⁰ and recovered after 7 days of incubation. 5 μl of virus was transduced into mouse neuroglia culture by PEI-mediated transfection. EGFP expression is indicated by arrows in FIG. 11A.

Recombinant AAV1-CBA-EGFP virus produced in embryonated eggs and recovered after 15 days of incubation. 2 μl of virus was transduced into mouse brain in vivo. EGFP expression was evaluated by IHC. Anti-EGFP histology is indicated by arrows in FIG. 11B.

These results validate the clinical value of recombinant AAV expressing transgenes of interest packaged and propagated in embryonated chicken eggs.

### Example 3. Chicken Embryonated Egg Production Methods Via Allantoic Inoculation

Next, the rHSV inoculation protocol was evaluated. That is, it was sought to validate that use of rHSV helper viruses in the packaging and propagation of AAV particles was compatible with AAV egg production and to investigate the biodistribution of rHSV-AAV particles in the embryonated chicken egg. In these experiments, in vitro inoculation of an allantoic vesicle encased in a chorioallantoic membrane (CAM), as extracted from an embryonated chicken egg and placed in a 10 cm Petri dish, served as a proxy for inoculation of the egg itself. rHSV-AAV vectors encoding humanized green fluorescent protein (hGFP) transgene under the control of a chicken beta actin (CBA) promoter and rHSV helper viruses containing a cassette containing the rep2 and cap2/cap9 genes from AAV were co-infected into six CAMs (labeled CAMs 1-6) extracted from ten day old embryonated chicken eggs. Inoculation was by injection through the chorioallantoic membrane (CAM) and into the allantoic cavity. rHSV-AAV was introduced at a MOI of 2, and rHSV-rep2capX was introduced at a MOI of 4. (rHSV-AAV-GFP was administered in an amount of 2.98×10⁸ plaque forming units (PFU) (or 59 μl per egg); rHSV-Rep2Cap2 was administered to CAMs 1-3 in an amount of 4.09×10⁸ PFU (86 μl per egg); and rHSV-Rep2Cap9 was administered to CAMs 4-6 in an amount of 1.91×10⁸ PFU (184 μl per egg).)

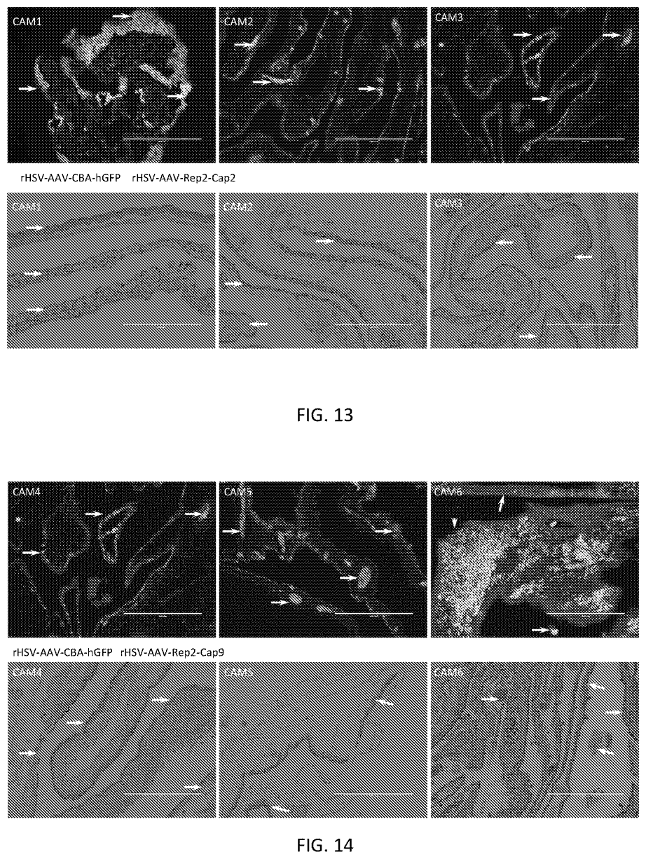

After 72 hours, allantoic fluid was harvested and used for rAAV virus purification. CAMs 1-6 were analyzed by direct fluorescence (FIGS. 13-14, top) and immunohistochemistry (FIGS. 13-14, bottom) for biodistribution of hGFP transgene expression. Rabbit secondary antibodies against GFP were used to detect the levels of rAAV genome (expressing hGFP) in the CAM. A DAB (3,3′-diaminobenzidine) stain was used to produce a brown-colored signal. A hematoxylin counter stain was used to show general layout and distribution of cells. Intensity of the stain corresponded to level of GFP expression of the protein, indicating that the AAV was distributed in the CAM. In particular, CAMs 1 and 6 showed high GFP expression. Measurement of viral load showed that inoculation with rHSV helper virus produced viral titers as high as 7.38×10¹⁰ vgs/ml, which corresponded to a viral yield per egg (per ovum) of 1.48×10¹⁰ vgs [(vgs/ml*0.2 ml)].

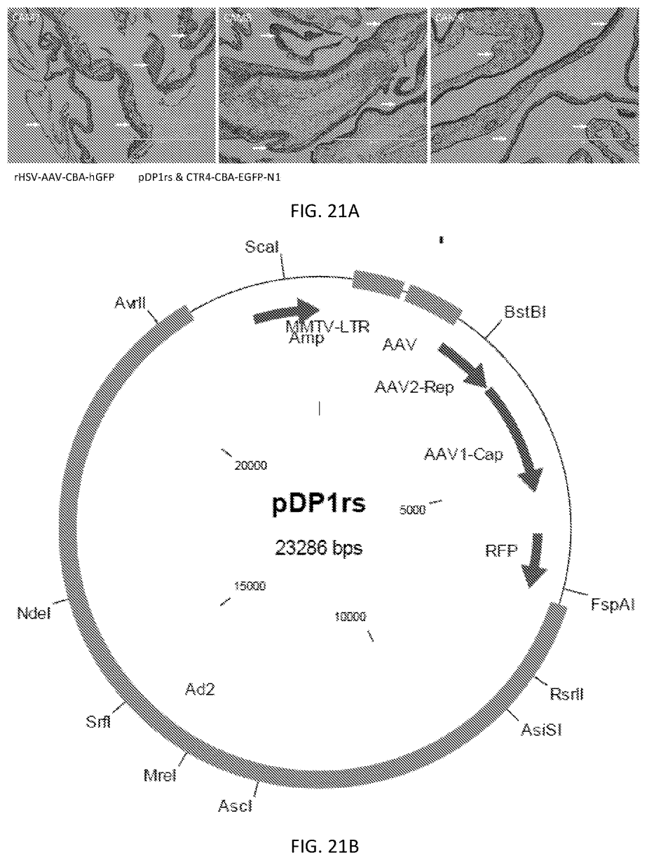

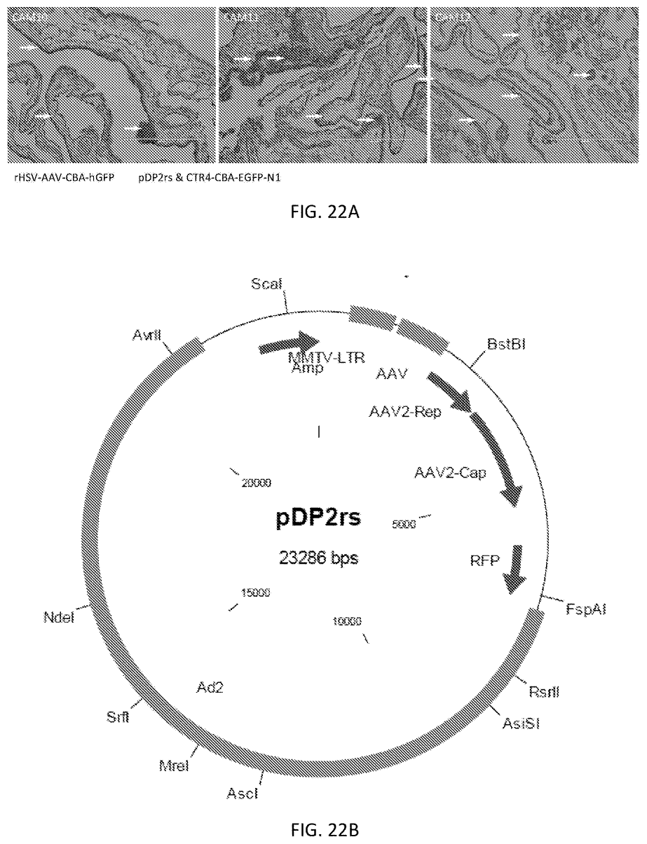

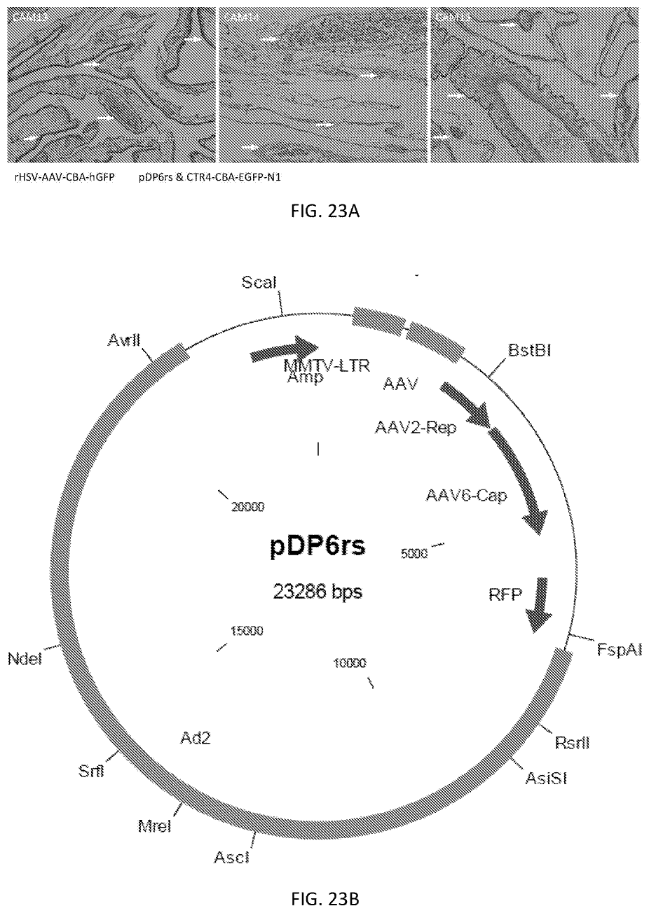

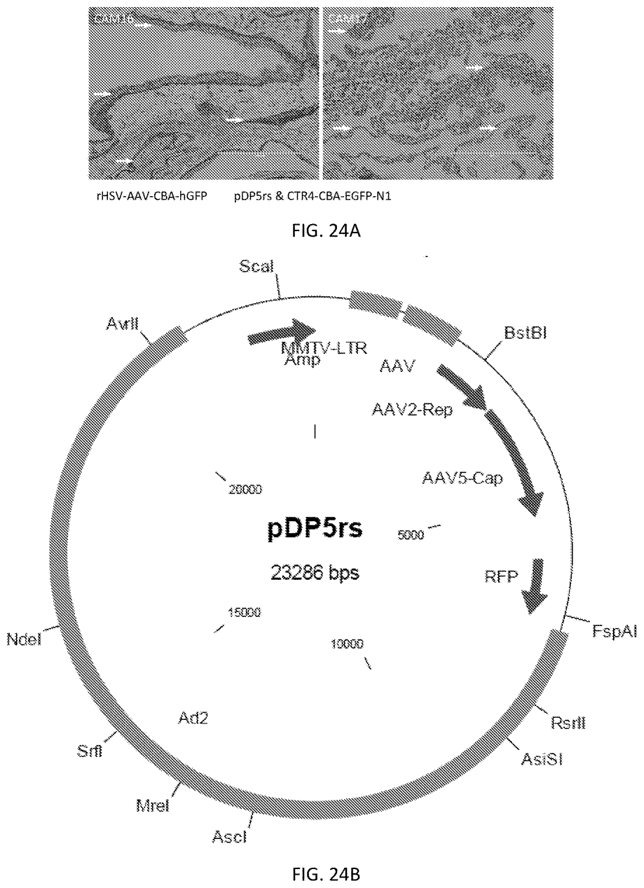

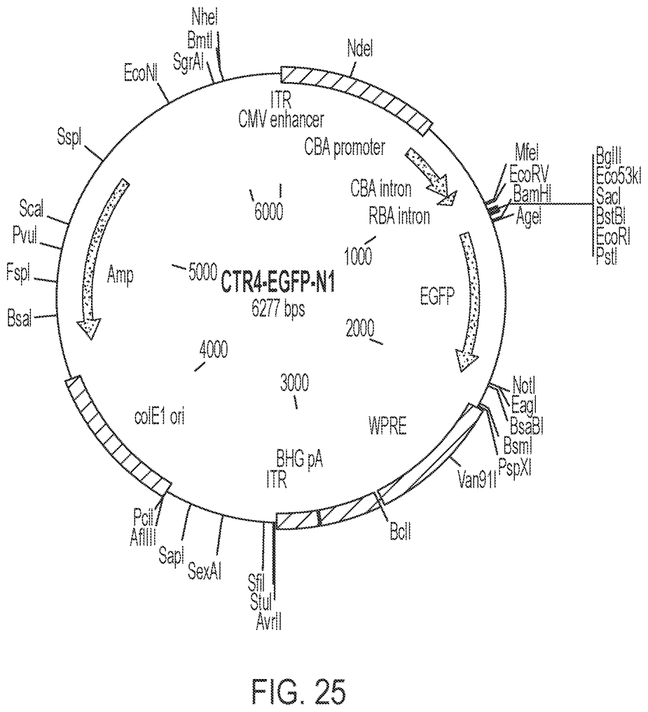

AAV virus production following a transfection protocol was also investigated in combination with AAV helper plasmids. A proprietary CTR4-CBA-EGFP-N1 vector (in an amount of 5.5 μg), an rHSV-AAV nucleic acid vector encoding the hGFP transgene, and one of four pDPrs helper plasmids (in an amount of 16.5 μg) were co-transfected using 244 μl PEI solution into eleven CAMs (CAMs numbered 7-17) extracted from ten day old embryonated chicken eggs. (Here, no rHSV vectors encoding AAV rep or cap were introduced to distinguish this protocol from the rHSV inoculation protocol.) A plasmid map for CTR4-CBA-EGFP-N1 is depicted in FIG. 25, and a nucleotide sequence for this plasmid is provided below. This vector contains an enhanced GFP transgene under the control of a CBA promoter and a CMV enhancer; it also contains a WPRE element and a bovine growth hormone (bGH) polyA tail. The four helper plasmids evaluated were pDP1rs, pDP2rs, pDP5rs, and pDP6rs (all purchased from PlasmidFactory®). Plasmid maps for each of these four helpers are depicted in FIGS. 21B, 22B, 23B, and 24B. These helper plasmids each contain an expression cassette encoding reporter RFP, capX, repX, and the adenoviral helper genes.

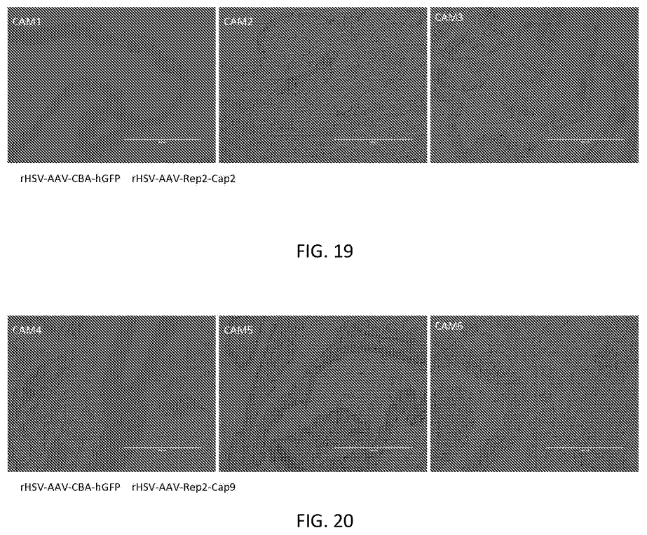

Direct fluorescence (FIGS. 15-18, top) and immunohistochemistry (FIGS. 15-18, bottom) was evaluated to determine GFP expression were performed as described above. Results show that AAV virus was distributed throughout the CAM (CAMs 7-17). To validate the positive contributions of the pDPrs helper plasmids, negative controls were generated by inoculating into six additional CAMs with rHSV-AAV-CBA-hGFP and rHSV-AAV-Rep2CapX plasmids, in the absence of any pDPrs helper. Allantoic fluid was harvested as above, and RFP expression was measured using a rabbit secondary antibody against RFP (the antibody was conjugated with horseradish peroxidase) and stained with DAB. These controls showed little to no intensity of DAB signal (FIGS. 19-20), thus confirming the importance of the helper pDPrs plasmids to successful AAV production in the transfection protocol.

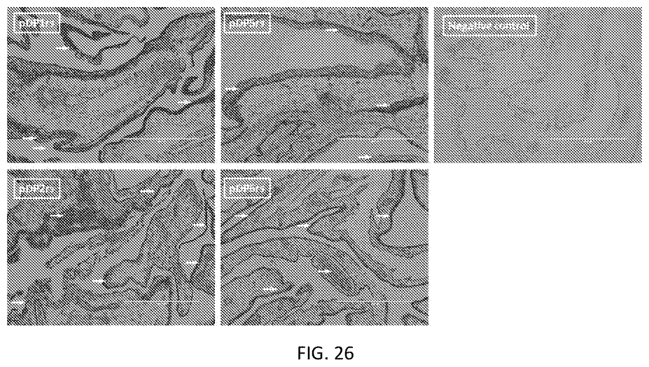

To further investigate helper plasmid transfection in AAV egg production, CAMs 7-17 were analyzed by immunohistochemistry for biodistribution of RFP expression, as contributed by the helper plasmids (see FIGS. 21A, 22A, 23A, 24A, and 26). The IHC results in a negative control is shown in FIG. 26 for comparison. RFP expression was measured using a rabbit secondary antibody against RFP and stained with DAB. The observed RFP expression pattern confirmed that capsid helper plasmids were necessary for high biodistribution of inoculated AAV vectors in the CAM. The intensity of the DAB signal corresponds to level of RFP expression. Little staining was observed in the negative control sample. Measurement of viral load showed that inoculation with AAV helper plasmids (pDPrs) produced viral titers as high as 6.37×10¹⁰ vgs/ml, which corresponded to a viral yield per egg (per ovum) of 1.27×10¹⁰ vgs.

These experiments indicate that inoculation protocols described herein—e.g., the rHSV and transfection inoculation protocols—provide for high AAV genome production levels in the allantoic cavities of embryonated avian eggs. Validations of these experiments using a scanning electron microscope are being conducted to demonstrate the successful packaging of rAAV genome into viral particles. Microscope experiments may confirm the biodistribution of packaged AAV viral particles in the CAMs of embryonated eggs.

The nucleotide sequence of the CTR4-CBA-EGFP-N1 transgene plasmid is provided below, as SEQ ID NO: 1. In some embodiments of the disclosed rAAV manufacturing methods, plasmids having a nucleotide sequence comprising at least 85%, at least 90%, at least 92.5%, at least 95%, at least 98%, or at least 99% sequence identity to SEQ ID NO: 1 are used. In some embodiments, a plasmid having the sequence of SEQ ID NO: 1 is used.

### REFERENCES

1. Vaccine. 2016 Oct. 26; 34(45): 5410-5413
2. Avian Diseases Vol. 33, No. 1 (1989), pp. 125-133
3. Cloning of an Avian Adeno-Associated Virus (AAAV) and Generation of Recombinant AAAV Particles. J Virol. 2003; 77(12): 6799-6810.
4. Lab Invest. 2005; 85(6):747-55
5. International Journal of Poultry Science 2007 6(11): 776-783
6. Transgenic Res. 1995 May; 4(3):192-8.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. A method of producing recombinant AAV (rAAV) comprising:
i) inoculating an embryonated avian egg with a first nucleic acid vector comprising a transgene and a second nucleic acid vector comprising AAV rep and cap genes,
ii) incubating the egg, and
iii) isolating rAAV from the egg,
wherein the rAAV is of non-avian origin.

2. The method of claim 1, wherein the avian egg is a chicken (Gallus gallus) egg.

3. The method of claim 1 or 2, wherein the isolated rAAV is substantially free of avian viruses.

4. The method of any one of claims 1-3, wherein the step of inoculating further comprises providing the avian egg with one or more helper genes.

5. The method of claim 4, wherein the helper genes are provided in the second nucleic acid vector, optionally wherein the helper genes comprise E1, E2, E4 and VA genes.

6. The method of claim 4, wherein the helper genes are provided in a third nucleic acid vector, optionally wherein the helper genes comprise E1, E2, E4 and VA genes.

7. The method of any one of the preceding claims, wherein the AAV is of mammalian origin.

8. The method of any one of the preceding claims, wherein the first and second nucleic acid vectors are combined with a cationic polymer prior to inoculation.

9. The method of claim 8, wherein the cationic polymer is polyethylenimine (PEI).

10. The method of any one of the preceding claims, wherein the egg is incubated for a period of at least 15 hours, at least 20 hours, at least 24 hours, at least 36 hours, at least 54 hours, at least 60 hours, at least 72 hours, at least 80 hours, or at least 90 hours.

11. The method of claim 10, wherein the egg is incubated for about 72 hours.

12. The method of any one of the preceding claims, wherein the rAAV is isolated using a manual pipette or syringe or a machine-controlled pipette or syringe.

13. The method of any one of the preceding claims, wherein the AAV is of an AAV1, an AAV2, an AAV3, an AAV4, an AAV5, an AAV6, an AAV7, an AAV8, an AAV9, an AAVrh10, an AAV12, or an AAV13 serotype.

14. The method of any one of the preceding claims, wherein the AAV is of an AAV1-M3, an AAV2-M3, an AAV2(tripYF), an AAV2(quadYF), an AAV2(pentaYF), an AAV2-BCDG(T491V+K556R), an AAV5-M2, an AAV5(Y719F), an AAV6(T492V+S663V), an AAV6(T492V+Y705F+Y731F), an AAV6(S551V+S663V), an AAV8-C&G(T494V), an AAV8-M3, an AAV8(Y733F), an AAV8(T494V+Y733F), AAV8(Y275F+Y447F+Y733F) or an AAV9-PHP.B serotype; or the serotype of another capsid variant.

15. The method of any one of the preceding claims, wherein an allantoic cavity, a chorioallantoic membrane, a yolk sac, or an amnion of the egg is inoculated.

16. The method of claim 15, wherein the allantoic cavity or the chorioallantoic membrane of the egg is inoculated.

17. The method of any one of the preceding claims further comprising:
iv) producing purified rAAV by subjecting the isolated rAAV to an iodixanol gradient.

18. The method of claim 17 further comprising:
v) inoculating an embryonated avian egg or an avian embryonic fibroblast (AEF) with the purified rAAV, and
vi) propagating the rAAV by incubating the avian egg or AEF, and isolating the rAAV from the egg or AEF.

19. The method of claim 18 further comprising:
vii) further purifying and/or concentrating the purified rAAV by tangential flow filtration and/or centrifugation, thereby producing further purified and/or concentrated rAAV.

20. The method of claim 18 or 19, wherein the egg is incubated for about 5 days, about 6 days, about 7 days, about 8 days, about 9 days, about 10 days, about 11 days, about 12 days, about 13 days, or about 15 days.

21. The method of any one of claims 18-20, wherein the egg is a chicken egg.

22. The method of claim 18 or 19, wherein the AEF is a chicken embryonic fibroblast (CEF).

23. The method of any one of claims 18-22, wherein the rAAV is further propagated in mammalian cells or insect cells.

24. The method of claim 23, wherein the mammalian cells are HEK 293T cells, BHK cells, or HeLa cells.

25. The method of any one of the preceding claims, wherein the egg is incubated in a shaker, a spinner or an automatic eggs incubator.

26. The method of any one of claims 18-24, wherein the AEF, mammalian cells and/or insect cells are incubated in a shaker flask, a spinner flask, a cellbag, or a bioreactor.

27. The method of any one of claims 17-26, wherein the purified rAAV is isolated at a titer of at least about 1×10⁸, at least about 5×10⁸, at least about 1×10⁹, at least about 5×10⁹, at least about 1×10¹⁰, at least about 5×10¹⁰, at least about 1×10¹¹, at least 2×10¹¹, or at least 5×10¹¹ vector genomes (vg)/ml.

28. The method of any one of claims 17-27, wherein the purified rAAV is isolated from a single egg at a titer of at least about 5×10¹⁰ vector genomes (vg)/ml.

29. The method of any one of the preceding claims, wherein the transgene encodes a therapeutic protein.

30. The method of claim 29, wherein the therapeutic protein is therapeutic for a lysosomal storage disease.

31. The method of claim 29, wherein the therapeutic protein is therapeutic for a neurological disability, a neuromotor deficit, or a neuroskeletal impairment.

32. The method of claim 29, wherein the therapeutic protein is therapeutic for a muscular disability, myopathy or cardiomyopathy.

33. The method of any one of claims 16-32, wherein the purified rAAV or the further purified and/or concentrated rAAV is added to a pharmaceutical composition.

34. The method of any one of claims 1-5 and 7-32, wherein the second nucleic acid vector is a helper plasmid selected from pDM, pDG, pDP1rs, pDP2rs, pDP3rs, pDP4rs, pDP5rs, and pDP6rs.

35. A pharmaceutical composition comprising the purified rAAV or the further purified and/or concentrated rAAV produced by the method of any one of claims 16-34, and a pharmaceutically acceptable carrier.

36. An rAAV particle comprising the purified rAAV or further purified and/or concentrated rAAV produced by the method of any one of claims 16-34.

37. A method of treatment comprising administering the composition of claim 35 or the rAAV particle of claim 36 to a subject in need thereof.

38. The method of claim 37, wherein the subject is a human.

39. An embryonated avian egg comprising a recombinant AAV of mammalian origin, wherein the recombinant AAV comprises a transgene.

40. The avian egg of claim 39, wherein the egg is adapted for production of isolated rAAV at a titer of a titer of at least 1×10⁸, at least 5×10⁸, at least 1×10⁹, at least 5×10⁹, at least 1×10¹⁰, at least 5×10¹⁰, at least about 1×10¹¹, at least 2×10¹¹, or at least 5×10¹¹ vector genomes (vg)/ml.

41. The avian egg of claim 39, wherein the egg is adapted for production of isolated rAAV at a titer of at least 5×10¹⁰ vector genomes (vg)/ml.

42. The avian egg of any one of claims 39-41, wherein the transgene encodes a therapeutic peptide.

43. The avian egg of any one of claims 39-42, wherein the rAAV is of an AAV1, an AAV2, an AAV3, an AAV4, an AAVS, an AAV6, an AAV7, an AAV8, an AAV9, an AAVrh10, an AAV12, or an AAV13 serotype.

44. The avian egg of any one of claims 39-43, wherein the rAAV is of an AAV1-M3, an AAV2-M3, an AAV2(tripYF), an AAV2(quadYF), an AAV2(pentaYF), an AAV2-BCDG(T491V+K556R), an AAVS-M2, an AAVS(Y719F), an AAV6(T492V+S663V), an AAV6(T492V+Y705F+Y731F), an AAV6(S551V+S663V), an AAV8-C&G(T494V), an AAV8-M3, an AAV8(Y733F), an AAV8(T494V+Y733F), an AAV8(Y275F+Y447F+Y733F) or an AAV9-PHP.B serotype; or the serotype of another capsid variant.

45. A method of producing recombinant AAV (rAAV) comprising:
i) inoculating an embryonated avian egg with a first recombinant herpes simplex virus (rHSV) comprising a nucleic acid comprising a transgene and a second rHSV virus comprising AAV rep and cap genes,
ii) incubating the egg, and
iii) isolating rAAV from the egg,
wherein the rAAV is of non-avian origin.

46. The method of claim 45, wherein the avian egg is a chicken (Gallus gallus) egg.

47. The method of claim 45 or 46, wherein the isolated rAAV is substantially free of avian viruses.

48. The method of any one of claims 45-47, wherein the HSV is of mammalian origin.

49. The method of any one of claims 45-48, wherein the egg is incubated for a period of at least 15 hours, at least 20 hours, at least 24 hours, at least 36 hours, at least 54 hours, at least 60 hours, at least 72 hours, at least 80 hours, or at least 90 hours.

50. The method of claim 49, wherein the egg is incubated for about 72 hours.

51. The method of any one of claims 45-50, wherein the rAAV is isolated using a manual pipette or syringe or a machine-controlled pipette or syringe.

52. The method of any one of claims 45-51, wherein an allantoic cavity, a chorioallantoic membrane, a yolk sac, an amnion or an embryo of the egg is inoculated.

53. The method of any one of claims 45-52, wherein the allantoic cavity or the chorioallantoic membrane of the egg is inoculated.

54. The method of claim 53, wherein the allantoic cavity is inoculated.

55. A method of producing recombinant AAV (rAAV) comprising:
i) inoculating an embryonated avian egg or an avian embryonic fibroblast (AEF) with an rAAV particle comprising a nucleic acid vector comprising a transgene, and
ii) propagating the rAAV particle by incubating the avian egg or AEF, and
iii) isolating rAAV from the egg or AEF,
wherein the rAAV is of non-avian origin.

56. The method of claim 55 further comprising:
iv) purifying and/or concentrating the isolated rAAV by tangential flow filtration and/or centrifugation, thereby producing purified and/or concentrated rAAV.

57. The method of claim 55 or 56, wherein the egg is incubated for about 5 days, about 6 days, about 7 days, about 8 days, about 9 days, about 10 days, about 11 days, about 12 days, about 13 days, or about 15 days.

58. The method of any one of claims 55-57, wherein the egg is a chicken egg.

59. The method of claim 57 or 58, wherein the AEF is a chicken embryonic fibroblast (CEF).

60. The method of any one of claims 55-59, wherein the rAAV is further propagated in mammalian cells or insect cells.

61. The method of claim 60, wherein the mammalian cells are HEK 293T cells, BHK cells, or HeLa cells.

62. The method of any one of claims 55-61, wherein the egg is incubated in a shaker, a spinner or an automatic eggs incubator.

63. The method of any one of claims 59-62, wherein the AEF, mammalian cells and/or insect cells are incubated in a shaker flask, a spinner flask, a cellbag, or a bioreactor.

64. A method of producing recombinant AAV (rAAV) comprising:
i) inoculating an embryonated avian egg with a first recombinant chicken embryo lethal orphan (rCELO) viral particle comprising a transgene and a second rCELO viral particle comprising AAV rep and cap genes,
ii) incubating the egg, and
iii) isolating rAAV from the egg,
wherein the rAAV is of non-avian origin.

65. A method of producing recombinant AAV (rAAV) comprising:
i) inoculating an embryonated avian egg with a first recombinant mammalian adenovirus comprising a nucleic acid comprising a transgene and a second mammalian adenovirus comprising AAV rep and cap genes,
ii) incubating the egg, and
iii) isolating rAAV from the egg,
wherein the rAAV is of non-avian origin.


SEQUENCE LISTING (1 sequence)

SEQ ID NO: 1 - DNA, 6277 bp, Artificial Sequence
  Synthetic
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtaccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaagaattggatcaaagcttcaattggatatcactagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattcctcgagcagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattc